(19) United States
(12) Patent Application Publication
(10) Pub. No.: US 2018/0243094 A1
(43) Pub. Date: Aug. 30, 2018

(54) Features for Implants with a Reduced Volumetric Density of Surface Roughness

(71) Applicant: HD LifeSciences LLC, Stoneham, MA (US)

(72) Inventors: Christopher L. Jones, Malden, MA (US); Ian Helmar, Beverly, MA (US); Lucas Diehl, Beverly, MA (US); Jason Tinley, Fort Worth, TX (US); Kevin D. Chappuis, Malden, MA (US); John F. Sullivan, Pelham, NH (US)

(21) Appl. No.: 15/903,667

(22) Filed: Feb. 23, 2018

Related U.S. Application Data
(60) Provisional application No. 62/480,383, filed on Apr. 1, 2017.
(60) Provisional application No. 62/480,391, filed on Apr. 1, 2017.
(60) Provisional application No. 62/619,260, filed on Jan. 19, 2018.
(60) Provisional application No. 62/463,089, filed on Feb. 24, 2017.

(51) Int. Cl. A61F 2/28; A61F 2/00; B22F 3/105

(52) U.S. Cl. CPC A61F 2/28; A61F 2/0077; A61L 27/54; A61L 2400/18; B22F 3/1055

(57) ABSTRACT

The invention disclosed herein includes implant features that can be used, in some embodiments, on devices with a volumetric density of less than about 100 percent and devices with a surface roughness of some value. The implant features include one or more protrusions mounted on the forward edge of an implant that can ease the distraction of tissue during implantation and reduce the occurrence of damage during a manufacturing process. In some embodiments, the protrusions have gaps in a non-axial direction with respect to the implant to allow axial compression with respect to the protrusions. In some embodiments, the protrusions have a circumferential gap between them and a body of a device to reduce any impact on the device's elastic modulus.

### CROSS REFERENCE TO RELATED APPLICATIONS

This application claims the benefit of U.S. Provisional Patent Application No. 62/463,089 filed Feb. 24, 2017, U.S. Provisional Patent Application No. 62/480,383 filed Apr. 1, 2017, U.S. Provisional Patent Application No. 62/480,391 filed Apr. 1, 2017, and U.S. Provisional Patent Application No. 62/619,260 filed Jan. 19, 2018, which are hereby incorporated by reference in their entirety.

### FIELD OF THE INVENTION

The present invention relates to implant features and the design and manufacture of implants with a reduced volumetric density and, in particular, to implant features and a method of using an additive process to manufacture implants with a lattice structure.

### BACKGROUND OF THE INVENTION

Medical implants with porous or open cell structures are useful for providing a scaffold for bone or tissue growth. Existing methods of manufacturing implants with porous or open cell structures include the use of additive processes, such as direct metal laser sintering (hereinafter “DMLS”) and selective laser sintering (hereinafter “SLS”). DMLS and SLS are similar in that they are capable of producing an object by using a power source (a laser) to sinter or melt layers of powdered material. The layers of material are generally built on a substantially flat platform or bed (hereinafter “platform”) and each layer can overhang the previous layer by a certain amount. The first layer of material is sintered or attached directly to the platform to provide stability to the rest of the object during the additive process. When the object is complete, the bond between the first layer and the platform must be broken.

The use of DMLS, SLS or another additive process (hereinafter “additive process”) allow the manufacture of implants with intricate internal structures that would be difficult to replicate using traditional manufacturing methods. Despite the advantages of additive processes, as the surface porosity of an object increases or the volumetric density of the object's surface decreases, it becomes increasing difficult to break the bond between the platform and the first layer without damage after the manufacturing process is complete. When an additive process is used to manufacture an implant with a highly porous surface or a low volumetric density structure, the surface area of the implant or outer layers of the structure attached to the platform are likely to be damaged during removal.

Therefore, there is a need for a method of designing and manufacturing implants with a reduced volumetric density without damaging or deforming portions of the surface or structure.

### BRIEF SUMMARY OF THE INVENTION

The present invention provides implant features and a method of designing and manufacturing implants using an additive process that avoids damage when removing the implant from a build surface of an additive process machine. The build surface of an additive process machine can be the build platform itself or a support between the manufactured device and the build platform. When used herein, a build surface can refer to the build platform or any intermediate surface between the build platform and the manufactured device. The inventive method involves designing an implant and build orientation with a portion of increased volumetric density in contact with the build surface. In some embodiments, the contact area between a device and a build surface is reduced to provide easier detachment after the additive process is complete.

The invention disclosed herein includes implant features that can be used, in some embodiments, on devices with a volumetric density of less than about 100 percent and devices with a surface roughness of some value. The implant features include one or more protrusions mounted on the forward edge of an implant that can ease the distraction of tissue during implantation and reduce the occurrence of damage during a manufacturing process. In some embodiments, the protrusions have gaps in a non-axial direction with respect to the implant to allow axial compression with respect to the protrusions. In some embodiments, the protrusions have a circumferential gap between them and a body of a device to reduce any impact on the device's elastic modulus.

### BRIEF DESCRIPTION OF THE SEVERAL VIEWS OF THE DRAWINGS

FIG. A1 is an isometric view of a single modified rhombic dodecahedron unit cell containing a full modified rhombic dodecahedron structure along with radial struts that comprise portions of adjacent unit cells.

FIG. A2 is a side view of a single modified rhombic dodecahedron unit cell showing the configuration of interconnections when viewed from a lateral direction.

FIG. A3 is a side view of a single modified rhombic dodecahedron unit cell where the central void is being measured using the longest dimension method.

FIG. A4 is a side view of a single modified rhombic dodecahedron unit cell where an interconnection is being measured using the longest dimension method.

FIG. A5 is a side view of the central void of a modified rhombic dodecahedron unit cell being measured with the largest sphere method.

FIG. A6 is a view from a direction normal to the planar direction of an interconnection being measured with the largest sphere method.

FIG. A7 is an isometric view of a single radial dodeca-rhombus unit cell.

FIG. A8 is a side view of a single radial dodeca-rhombus unit cell.

FIG. A9 is an isometric view of an example of a single node and single strut combination that could be used in a radial dodeca-rhombus unit cell.

FIG. A10 is a side view of an example of a single node and single strut combination that could be used in a radial dodeca-rhombus unit cell.

FIG. A11 is a side view of a single node and single strut combination configured for use in a lattice with an elastic modulus of approximately 3 GPa, viewed from the corner of the volume defining the bounds of the combination.

FIG. A12 is a side view of a single node and single strut combination configured for use in a lattice with an elastic modulus of approximately 4 GPa, viewed from the corner of the volume defining the bounds of the combination.

FIG. A13 is a side view of a single node and single strut combination configured for use in a lattice with an elastic modulus of approximately 10 GPa, viewed from the corner of the volume defining the bounds of the combination.

FIG. A14 is a side view of a single node and two adjacent struts viewed from the corner of the volume defining the bounds of the combination and the lateral separation angle.

FIG. A15 is an isometric view of a sub-unit cell comprised of a single node and four struts.

FIG. A16 is an isometric view of two sub-unit cells in a stacked formation where the upper sub-unit cell is inverted and fixed to the top of the lower sub-unit cell.

FIG. A17 is an isometric view of eight sub-unit cells stacked together to form a single unit cell.

FIG. 1 is a front view of a first exemplary embodiment of the invention showing leading-edge features to aid in distraction without increasing the bulk elastic modulus.

FIG. 2 is an upper lateral view of a first exemplary embodiment of the invention showing the leading-edge features and the configuration of the upper endplate.

FIG. 3 is an upper lateral sectioned view of a first embodiment of the invention showing the configuration of the leading-edge, including its substantially horizontal gap and circumferential gap.

FIG. 4 is a side sectioned view of a first embodiment of the invention also showing the configuration of the leading-edge, including its substantially horizontal gap and circumferential gap.

FIG. 5 is a side view of a first embodiment of the invention showing the configuration of the lead edge features and endplates.

FIG. 6 is a top sectioned view of a first exemplary embodiment of the invention showing the configuration of the circumferential gap behind the nose.

FIG. 6A is a top sectioned view of the lower nose with measurements of the leading edge comprising a circular sector.

FIG. 6B is a top sectioned view of an alternative lower nose shape.

FIG. 6C is a top sectioned view of another alternative lower nose shape.

FIG. 7 is an isometric view of a second exemplary embodiment of the invention showing an alternative configuration for an impact rail.

FIG. 8 is a side view of a second exemplary embodiment of the invention showing an alternative configuration for the leading-edge features, endplates and impact rail.

FIG. 9 is a side view of a third exemplary embodiment of the invention showing an alternative configuration for the leading-edge features and impact rail.

FIG. 10 is a side view of a fourth exemplary embodiment of the invention showing another alternative configuration for the leading-edge features and impact rail.

FIG. 11 is a side view of a first exemplary embodiment of an implant designed using the method of the present invention prior to removal from a build surface.

FIG. 12 is a side sectioned view of a first exemplary embodiment of an implant designed using the method of the present invention prior to removal from a build surface.

FIG. 13 is a perspective view of a first exemplary embodiment of an implant designed using the method of the present invention in its build orientation.

FIG. 14 is a perspective sectioned view of a first exemplary embodiment of an PLIF/TLIF implant designed using the method of the present invention in its build orientation.

FIG. 15 is a top sectioned view of a first exemplary embodiment of an implant designed using the method of the present invention prior to removal from a build surface.

FIG. 16 is a perspective view of a second exemplary embodiment of an implant designed using the method of the present invention after removal from a build surface.

### DETAILED DESCRIPTION OF THE INVENTION

In many situations, it is desirable to use an implant that is capable of bone attachment or osteointegration over time. It is also desirable in many situations to use an implant that is capable of attachment or integration with living tissue. Examples of implants where attachment to bone or osteointegration is beneficial include, but are not limited to, cervical, lumbar, and thoracic interbody fusion implants, vertebral body replacements, osteotomy wedges, dental implants, bone stems, acetabular cups, cranio-facial plating, bone replacement and fracture plating. In many applications, it is also desirable to stress new bone growth to increase its strength. According to Wolff's law, bone will adapt to stresses placed on it so that bone under stress will grow stronger and bone that isn't stressed will become weaker.

In some aspects, the systems and methods described herein can be directed toward implants that are configured for osteointegration and stimulating adequately stressed new bone growth. Many of the exemplary implants of the present invention are particularly useful for use in situations where it is desirable to have strong bone attachment and/or bone growth throughout the body of an implant. Whether bone growth is desired only for attachment or throughout an implant, the present invention incorporates a unique lattice structure that can provide mechanical spacing, a scaffold to support new bone growth and a modulus of elasticity that allows new bone growth to be loaded with physiological forces. As a result, the present invention provides implants that grow stronger and healthier bone for more secure attachment and/or for a stronger bone after the implant osteointegrates.

The exemplary embodiments of the invention presented can be comprised, in whole or in part, of a lattice. A lattice, as used herein, refers to a three-dimensional material with one or more interconnected openings that allow a fluid to communicate from one location to another location through an opening. A three-dimensional material refers to a material that fills a three-dimensional space (i.e. has height, width and length). Lattices can be constructed by many means, including repeating various geometric shapes or repeating random shapes to accomplish a material with interconnected openings. An opening in a lattice is any area within the bounds of the three-dimensional material that is devoid of that material. Therefore, within the three-dimensional boundaries of a lattice, there is a volume of material and a volume that is devoid of that material.

The material that provides the structure of the lattice is referred to as the primary material. The structure of a lattice does not need to provide structural support for any purpose, but rather refers to the configuration of the openings and interconnections that comprise the lattice. An opening in a lattice may be empty, filled with a gaseous fluid, filled with a liquid fluid, filled with a solid or partially filled with a fluid and/or solid. Interconnections, with respect to openings, refer to areas devoid of the primary material and that link at least two locations together. Interconnections may be configured to allow a fluid to pass from one location to another location.

A lattice can be defined by its volumetric density, meaning the ratio between the volume of the primary material and the volume of voids presented as a percentage for a given three-dimensional material. The volume of voids is the difference between the volume of the bounds of the three-dimensional material and the volume of the primary material. The volume of voids can comprise of the volume of the openings, the volume of the interconnections and/or the volume of another material present. For example, a lattice with a 30% volumetric density would be comprised of 30% primary material by volume and 70% voids by volume over a certain volume. A lattice with a 90% volumetric density would be comprised of 90% primary material by volume and 10% voids by volume over a certain volume. In three-dimensional materials with a volumetric density of less than 50%, the volume of the primary material is less than the volume of voids. While the volumetric density refers to the volume of voids, the voids do not need to remain void and can be filled, in whole or in part, with a fluid or solid prior to, during or after implantation.

Lattices comprised of repeating geometric patterns can be described using the characteristics of a repeating unit cell. A unit cell in a repeating geometric lattice is a three-dimensional shape capable of being repeated to form a lattice. A repeating unit cell can refer to multiple identical unit cells that are repeated over a lattice structure or a pattern through all or a portion of a lattice structure. Each unit cell is comprised of a certain volume of primary material and a certain void volume, or in other words, a spot volumetric density. The spot volumetric density may cover as few as a partial unit cell or a plurality of unit cells. In many situations, the spot volumetric density will be consistent with the material's volumetric density, but there are situations where it could be desirable to locally increase or decrease the spot volumetric density.

Unit cells can be constructed in numerous volumetric shapes containing various types of structures. Unit cells can be bound by a defined volume of space to constrict the size of the lattice structure or other type of structure within the unit cell. In some embodiments, unit cells can be bound by volumetric shapes, including but not limited to, a cubic volume, a cuboid volume, a hexahedron volume or an amorphous volume. The unit cell volume of space can be defined based on a number of faces that meet at corners. In examples where the unit cell volume is a cubic, cuboid or hexahedron volume, the unit cell volume can have six faces and eight corners, where the corners are defined by the location where three faces meet. Unit cells may be interconnected in some or all areas, not interconnected in some or all areas, of a uniform size in some or all areas or of a nonuniform size in some or all areas. In some embodiments disclosed herein that use a repeating geometric pattern, the unit cells can be defined by a number of struts defining the edges of the unit cell and joined at nodes about the unit cell. Unit cells so defined can share certain struts among more than one unit cell, so that two adjacent unit cells may share a common planar wall defined by struts common to both cells. In some embodiments disclosed herein that use a repeating geometric pattern, the unit cells can be defined by a node and a number of struts extending radially from that node.

While the present application uses volumetric density to describe exemplary embodiments, it is also possible to describe them using other metrics, including but not limited to cell size, strut size or stiffness. Cell size may be defined using multiple methods, including but not limited to cell diameter, cell width, cell height and cell volume. Strut size may be defined using multiple methods, including but not limited to strut length and strut diameter.

Repeating geometric patterns are beneficial for use in lattice structures contained in implants because they can provide predictable characteristics. Many repeating geometric shapes may be used as the unit cell of a lattice, including but are not limited to, rhombic dodecahedron, diamond, dodecahedron, square, pentagonal, hexagonal, octagonal, sctet struts, trunic octa, diagonal struts, other known geometric structures, and rounded, reinforced, weakened, or simplified versions of each geometry.

Lattices may also be included in implants as a structural component or a nonstructural component. Lattices used in structural applications may be referred to herein as structural lattices, load-bearing lattices or stressed lattices. In some instances, structural lattices, load-bearing lattices or stressed lattices may be simply referred to as a lattice. Repeating geometric shaped unit cells, particularly the rhombic dodecahedron, are well suited, in theory, for use in structural lattices because of their strength to weight ratio. To increase the actual strength and fatigue resistance of a rhombic dodecahedron lattice, the present invention, in some embodiments, includes a modified strut comprised of triangular segments, rather than using a strut with a rectangular or circular cross section. Some embodiments herein also modify the angles defining the rhombic faces of a rhombic dodecahedron to change the lattice's elastic modulus and fatigue resistance. The use of triangular segments provides a lattice with highly predictable printed properties that approach the theoretical strength values for a rhombic dodecahedron lattice.

In structural lattice applications, the strength and elastic modulus of the lattice can be approximated by the volumetric density. When the volumetric density increases, the strength and the elastic modulus increases. Compared to other porous structures, the lattice of the present invention has a higher strength and elastic modulus for a given volumetric density because of its ability to use the high strength to weight benefits of a rhombic dodecahedron, modified rhombic dodecahedron or radial dodeca-rhombus unit cell.

When configured to provide support for bone or tissue growth, a lattice may be referred to as a scaffold. Lattices can be configured to support bone or tissue growth by controlling the size of the openings and interconnections disposed within the three-dimensional material. A scaffold, if used on the surface of an implant, may provide an osteointegration surface that allows adjacent bone to attach to the implant. A scaffold may also be configured to provide a path that allows bone to grow further than a mere surface attachment. Scaffolds intended for surface attachment are referred to herein as surface scaffolds. A surface scaffold may be one or more unit cells deep, but does not extend throughout the volume of an implant. Scaffolds intended to support in-growth beyond mere surface attachment are referred to herein as bulk scaffolds. Scaffolds may also be included in implants as a structural component or a nonstructural component. Scaffolds used in structural applications may be referred to herein as structural scaffolds, load-bearing scaffolds or stressed scaffolds. In some instances, structural scaffolds, load-bearing scaffolds or stressed scaffolds may be simply referred to as a scaffold. In some instances, the use of the term scaffold may refer to a material configured to provide support for bone or tissue growth, where the material is not a lattice.

The scaffolds described herein can be used to promote the attachment or in-growth of various types of tissue found in living beings. As noted earlier, some embodiments of the scaffold are configured to promote bone attachment and in-growth. The scaffolds can also be configured to promote attachment of in-growth of other areas of tissue, such as fibrous tissue. In some embodiments, the scaffold can be configured to promote the attachment or in-growth of multiple types of tissue. Some embodiments of the scaffolds are configured to be implanted near or abutting living tissue. Near living tissue includes situations where other layers, materials or coatings are located between a scaffold and any living tissue.

In some embodiments, the present invention uses bulk scaffolds with openings and interconnections that are larger than those known in the art. Osteons can range in diameter from about 100 μm and it is theorized that a bundle of osteons would provide the strongest form of new bone growth. Bone is considered fully solid when it has a diameter of greater than 3 mm so it is theorized that a bundle of osteons with a diameter equaling approximately half of that value would provide significant strength when grown within a scaffold. It is also theorized that osteons may grow in irregular shapes so that the cross-sectional area of an osteon could predict its strength. A cylindrical osteon growth with a 3 mm diameter has a cross-sectional area of approximately 7 square mm and a cylindrical osteon with a 1.5 mm diameter has a cross-sectional area of 1.8 square mm. It is theorized that an osteon of an irregular shape with a cross-sectional area of at least 1.8 square millimeters could provide a significant strength advantage when grown in a scaffold.

Most skilled in the art would indicate that pores or openings with a diameter or width between 300 μm to 900 μm, with a pore side of 600 μm being ideal, provide the best scaffold for bone growth. Instead, some embodiments of the present invention include openings and interconnections with a diameter or width on the order of 1.0 to 15.0 times the known range, with the known range being 300 μm to 900 μm, resulting in openings from 0.07 mm² up to 145 mm² cross sectional area for bone growth. In some examples, pores or openings with a diameter or width between and including 100 μm to 300 μm could be beneficial. Some examples include openings and interconnections with a diameter on the order of 1.0 to 5.0 times the known range. It has been at least theorized that the use of much larger openings and interconnections than those known in the art will allow full osteons and solid bone tissue to form throughout the bulk scaffold, allowing the vascularization of new, loadable bone growth. In some examples, these pores may be 3 mm in diameter or approximately 7 mm² in cross sectional area. In other examples, the pores are approximately 1.5 mm in diameter or approximately 1.75 mm² in cross sectional area. The use of only the smaller diameter openings and interconnections known in the art are theorized to limit the penetration of new bone growth into a bulk scaffold because the smaller diameter openings restrict the ability of vascularization throughout the bulk scaffold.

A related structure to a lattice is a closed cell material. A closed cell material is similar to a lattice, in that it has openings contained within the bounds of a three-dimensional material, however, closed cell materials generally lack interconnections between locations through openings or other pores. A closed cell structure may be accomplished using multiple methods, including the filling of certain cells or through the use of solid walls between the struts of unit cells. A closed cell structure can also be referred to as a cellular structure. It is possible to have a material that is a lattice in one portion and a closed cell material in another. It is also possible to have a closed cell material that is a lattice with respect to only certain interconnections between openings or vice versa. While the focus of the present disclosure is on lattices, the structures and methods disclosed herein can be easily adapted for use on closed cell structures within the inventive concept.

The lattice used in the present invention can be produced from a range of materials and processes. When used as a scaffold for bone growth, it is desirable for the lattice to be made of a biocompatible material that allows for bone attachment, either to the material directly or through the application of a bioactive surface treatment. In one example, the scaffold is comprised of an implantable metal. Implantable metals include, but are not limited to, zirconium, stainless steel (316 & 316L), tantalum, nitinol, cobalt chromium alloys, titanium and tungsten, and alloys thereof. Scaffolds comprised of an implantable metal may be produced using an additive metal fabrication or 3D printing process. Appropriate production processes include, but are not limited to, direct metal laser sintering, selective laser sintering, selective laser melting, electron beam melting, laminated object manufacturing and directed energy deposition.

In another example, the lattice of the present invention is comprised of an implantable metal with a bioactive coating. Bioactive coatings include, but are not limited to, coatings to accelerate bone growth, anti-thrombogenic coatings, anti-microbial coatings, hydrophobic or hydrophilic coatings, and hemophobic, superhemophobic, or hemophilic coatings. Coatings that accelerate bone growth include, but are not limited to, calcium phosphate, hydroxyapatite (“HA”), silicate glass, stem cell derivatives, bone morphogenic proteins, titanium plasma spray, titanium beads and titanium mesh. Anti-thrombogenic coatings include, but are not limited to, low molecular weight fluoro-oligomers. Anti-microbial coatings include, but are not limited to, silver, organosilane compounds, iodine and silicon-nitride. Superhemophobic coatings include fluorinated nanotubes.

In another example, the lattice is made from a titanium alloy with an optional bioactive coating. In particular, Ti6Al4V ELI wrought (American Society for Testing and Materials (“ASTM”) F136) is a particularly well-suited titanium alloy for scaffolds. While Ti6Al4V ELI wrought is the industry standard titanium alloy used for medical purposes, other titanium alloys, including but not limited to, unalloyed titanium (ASTM F67), Ti6Al4V standard grade (ASTM F1472), Ti6Al7Nb wrought (ASTM 1295), Ti5Al2.5Fe wrought (British Standards Association/International Standard Organization Part 10), CP and Ti6Al4V standard grade powders (ASTM F1580), Ti13Nb13Zr wrought (ASTM F1713), the lower modulus Ti-24Nb-4Zr-8Sn and Ti12Mo6Zr2Fe wrought (ASTM F1813) can be appropriate for various embodiments of the present invention.

Titanium alloys are an appropriate material for scaffolds because they are biocompatible and allow for bone attachment. Various surface treatments can be done to titanium alloys to increase or decrease the level of bone attachment. Bone will attach to even polished titanium, but titanium with a surface texture allows for greater bone attachment. Methods of increasing bone attachment to titanium may be produced through a forging or milling process, sandblasting, acid etching, and the use of a bioactive coating. Titanium parts produced with an additive metal fabrication or 3D printing process, such as direct metal laser sintering, can be treated with an acid bath to reduce surface stress risers, normalize surface topography, and improve surface oxide layer, while maintaining surface roughness and porosity to promote bone attachment.

Additionally, Titanium or other alloys may be treated with heparin, heparin sulfate (HS), glycosaminoglycans (GAG), chondroitin-4-sulphate (C4S), chondroitin-6-sulphate (C6S), hyaluronan (HY), and other proteoglycans with or without an aqueous calcium solution. Such treatment may occur while the material is in its pre-manufacturing form (often powder) or subsequent to manufacture of the structure.

While a range of structures, materials, surface treatments and coatings have been described, it is believed that a lattice using a repeating modified rhombic dodecahedron (hereinafter “MRDD”) unit cell can present a preferable combination of stiffness, strength, fatigue resistance, and conditions for bone ingrowth. In some embodiments, the repeating MRDD lattice is comprised of titanium or a titanium alloy. A generic rhombic dodecahedron (hereinafter “RDD”), by definition, has twelve sides in the shape of rhombuses. When repeated in a lattice, an RDD unit cell is comprised of 24 struts that meet at 14 vertices. The 24 struts define the 12 planar faces of the structure and disposed at the center of each planar face is an opening, or interconnection, allowing communication from inside the unit cell to outside the unit cell.

An example of the MRDD unit cell B10 used in the present invention is shown in FIGS. A1-A5. In FIG. A1 is an isometric view of a single MRDD unit cell B10 containing a full MRDD structure along with radial struts that comprise portions of adjacent unit cells. In FIG. A2 is a side view of a single MRDD unit cell B10 showing the configuration of interconnections when viewed from a lateral direction. A top or bottom view of the MRDD unit cell B10 would be substantially the same as the side view depicted in FIG. A2. The MRDD unit cell B10 differs in both structural characteristics and method of design from generic RDD shapes. A generic RDD is comprised of 12 faces where each face is an identical rhombus with an acute angle of 70.5 degrees and an obtuse angle of 109.5 degrees. The shape of the rhombus faces in a generic RDD do not change if the size of the unit cell or the diameter of the struts are changed because the struts are indexed based on their axis and each pass through the center of the 14 nodes or vertices.

In some embodiments of the MRDD, each node is contained within a fixed volume that defines its bounds and provides a fixed point in space for the distal ends of the struts. The fixed volume containing the MRDD or a sub-unit cell of the MRDD can be various shapes, including but not limited to, a cubic, cuboid, hexahedron or amorphous volume. Some examples use a fixed volume with six faces and eight corners defined by locations where three faces meet. The orientation of the struts can be based on the center of a node face at its proximate end and the nearest corner of the volume to that node face on its distal end. Each node is preferably an octahedron, more specifically a square bipyramid (i.e. a pyramid and inverted pyramid joined on a horizontal plane). Each node, when centrally located in a cuboid volume, more preferably comprises a square plane parallel to a face of the cuboid volume, six vertices and is oriented so that each of the six vertices are positioned at their closest possible location to each of the six faces of the cuboid volume. Centrally located, with regards to the node's location within a volume refers to positioning the node at a location substantially equidistant from opposing walls of the volume. In some embodiments, the node can have a volumetric density of 100 percent and in other embodiments, the node can have a volumetric density of less than 100 percent. Each face of the square bipyramid node can be triangular and each face can provide a connection point for a strut.

The struts can also be octahedrons, comprising an elongate portion of six substantially similar elongate faces and two end faces. The elongate faces can be isosceles triangles with a first internal angle, angle A, and a second internal angle, angle B, where angle B is greater than angle A. The end faces can be substantially similar isosceles triangles to one another with a first internal angle, angle C, and a second internal angle, angle D, where angle D is greater than angle C. Preferably, angle C is greater than angle A.

The strut direction of each strut is a line or vector defining the orientation of a strut and it can be orthogonal or non-orthogonal relative to the planar surface of each node face. In the MRDD and radial dodeca-rhombus structures disclosed herein, the strut direction can be determined using a line extending between the center of the strut end faces, the center of mass along the strut or an external edge or face of the elongate portion of the strut. When defining a strut direction using a line extending between the center of the strut end faces, the line is generally parallel to the bottom face or edge of the strut. When defining a strut direction using a line extending along the center of mass of the strut, the line can be nonparallel to the bottom face or edge of the strut. The octahedron nodes of the MRDD can be scaled to increase or decrease volumetric density by changing the origin point and size of the struts. The distal ends of the struts, however, are locked at the fixed volume corners formed about each node so that their angle relative to each node face changes as the volumetric density changes. Even as the volumetric density of an MRDD unit cell changes, the dimensions of the fixed volume formed about each node does not change. In FIG. A1, dashed lines are drawn between the corners of the MRDD unit cell B10 to show the cube B11 that defines its bounds. In the MRDD unit cell in FIG. A1, the height B12, width B13 and depth B14 of the unit cell are substantially the same, making the area defined by B11 a cube.

In some embodiments, the strut direction of a strut can intersect the center of the node and the corner of the cuboid volume nearest to the node face where the strut is fixed. In some embodiments, the strut direction of a strut can intersect just the corner of the cuboid volume nearest to the node face where the strut is fixed. In some embodiments, a reference plane defined by a cuboid or hexahedron face is used to describe the strut direction of a strut. When the strut direction of a strut is defined based on a reference plane, it can be between 0 degrees and 90 degrees from the reference plane. When the strut direction of a strut is defined based on a reference plane, it is preferably eight degrees to 30 degrees from the reference plane.

By indexing the strut orientation to a variable node face on one end and a fixed point on its distal end, the resulting MRDD unit cell can allow rhombus shaped faces with a smaller acute angle and larger obtuse angle than a generic RDD. The rhombus shaped faces of the MRDD can have two substantially similar opposing acute angles and two substantially similar opposing obtuse angles. In some embodiments, the acute angles are less than 70.5 degrees and the obtuse angles are greater than 109.5 degrees. In some embodiments, the acute angles are between 0 degrees and 55 degrees and the obtuse angles are between 125 degrees and 180 degrees. In some embodiments, the acute angles are between 8 degrees and 60 degrees and the obtuse angles are between 120 degrees and 172 degrees. The reduction in the acute angles increases fatigue resistance for loads oriented across the obtuse angle corner to far obtuse angle corner. The reduction in the acute angles and increase in obtuse angles also orients the struts to increase the MRDD's strength in shear and increases the fatigue resistance. By changing the rhombus corner angles from a generic RDD, shear loads pass substantially in the axial direction of some struts, increasing the shear strength. Changing the rhombus corner angles from a generic RDD also reduces overall deflection caused by compressive loads, increasing the fatigue strength by resisting deflection under load.

When placed towards the center of a lattice structure, the 12 interconnections of a unit cell connect to 12 different adjacent unit cells, providing continuous paths through the lattice. The size of the central void and interconnections in the MRDD may be defined using the longest dimension method as described herein. Using the longest dimension method, the central void can be defined by taking a measurement of the longest dimension as demonstrated in FIG. A3. In FIG. A3, the longest dimension is labeled as distance AA. The distance AA can be taken in the vertical or horizontal directions (where the directions reference the directions on the page) and would be substantially the same in this example. The interconnections may be defined by their longest measurement when viewed from a side, top or bottom of a unit cell. In FIG. A4, the longest dimension is labeled as distance AB. The distance AB can be taken in the vertical or horizontal directions (where the directions reference the directions on the page). The view in FIG. A4 is a lateral view, however, in this example the unit cell will appear substantially the same when viewed from the top or bottom.

The size of the central void and interconnections can alternatively be defined by the largest sphere method as described herein. Using the largest sphere method, the central void can be defined by the diameter of the largest sphere that can fit within the central void without intersecting the struts. In FIG. A5 is an example of the largest sphere method being used to define the size of a central void with a sphere with a diameter of BA. The interconnections are generally rhombus shaped and their size can alternatively be defined by the size of the length and width of three circles drawn within the opening. Drawn within the plane defining a side, a first circle BB1 is drawn at the center of the opening so that it is the largest diameter circle that can fit without intersecting the struts. A second circle BB2 and third circle BB3 is them drawn so that they are tangential to the first circle BB1 and the largest diameter circles that can fit without intersecting the struts. The diameter of the first circle BB1 is the width of the interconnection and the sum of the diameters of all three circles BB1, BB2 & BB3 represents the length of the interconnection. Using this method of measurement removes the acute corners of the rhombus shaped opening from the size determination. In some instances, it is beneficial to remove the acute corners of the rhombus shaped opening from the calculated size of the interconnections because of the limitations of additive manufacturing processes. For example, if an SLS machine has a resolution of 12 μm where the accuracy is within 5 μm, it is possible that the acute corner could be rounded by the SLS machine, making it unavailable for bone ingrowth. When designing lattices for manufacture on less precise additive process equipment, it can be helpful to use this measuring system to better approximate the size of the interconnections.

Using the alternative measuring method, in some examples, the width of the interconnections is approximately 600 μm and the length of the interconnections is approximately 300 μm. The use of a 600 μm length and 300 μm width provides an opening within the known pore sizes for bone growth and provides a surface area of roughly 1.8 square millimeters, allowing high strength bone growth to form. Alternative embodiments may contain interconnections with a cross sectional area of 1.0 to 15.0 times the cross-sectional area of a pore with a diameter of 300 μm. Other embodiments may contain interconnections with a cross sectional area of 1.0 to 15.0 times the cross-sectional area of a pore with a diameter of 900 μm.

The MRDD unit cell also has the advantage of providing at least two sets of substantially homogenous pore or opening sizes in a lattice structure. In some embodiments, a first set of pores have a width of about 200 μm to 900 μm and a second set of pores have a width of about 1 to 15 times the width of the first set of pores. In some embodiments, a first set of pores can be configured to promote the growth of osteoblasts and a second set of pores can be configured to promote the growth of osteons. Pores sized to promote osteoblast growth can have a width of between and including about 100 μm to 900 μm. In some embodiments, pores sized to promote osteoblast growth can have a width that exceeds 900 μm. Pores sized to promote the growth of osteons can have a width of between and including about 100 μm to 13.5 mm. In some embodiments, pores sized to promote osteon growth can have a width that exceeds 13.5 mm.

In some embodiments, it is beneficial to include a number of substantially homogenous larger pores and a number of substantially homogenous smaller pores, where the number of larger pores is selected based on a ratio relative to the number of smaller pores. For example, some embodiments have one large pore for every one to 25 small pores in the lattice structure. Some embodiments preferably have one large pore for every eight to 12 smaller pores. In some embodiments, the number of larger and smaller pores can be selected based on a percentage of the total number of pores in a lattice structure. For example, some embodiments can include larger pores for four percent to 50 percent of the total number of pores and smaller pores for 50 percent to 96 percent of the total number of pores. More preferably, some embodiments can include larger pores for about eight percent to 13 percent of the total number of pores and smaller pores for about 87 percent to 92 percent of the total number of pores. It is believed that a lattice constructed with sets of substantially homogenous pores of the disclosed two sizes provides a lattice structure that simultaneously promotes osteoblast and osteon growth.

The MRDD unit cell may also be defined by the size of the interconnections when viewed from a side, top or bottom of a unit cell. The MRDD unit cell has the same appearance when viewed from a side, top or bottom, making the measurement in a side view representative of the others. When viewed from the side, as in FIG. A4, an MRDD unit cell displays four distinct diamond shaped interconnections with substantially right angles. The area of each interconnection is smaller when viewed in the lateral direction than from a direction normal to the planar direction of each interconnection, but the area when viewed in the lateral direction can represent the area available for bone to grow in that direction. In some embodiments, it may be desirable to index the properties of the unit cell and lattice based on the area of the interconnections when viewed from the top, bottom or lateral directions.

In some embodiments of the lattice structures disclosed herein, the central void is larger than the length or width of the interconnections. Because the size of each interconnection can be substantially the same in a repeating MRDD structure, the resulting lattice can be comprised of openings of at least two discrete sizes. In some embodiments, it is preferable for the diameter of the central void to be approximately two times the length of the interconnections. In some embodiments, it is preferable for the diameter of the central void to be approximately four times the width of the interconnections.

In some embodiments, the ratio between the diameter of the central void and the length or width of the interconnections can be changed to create a structural lattice of a particular strength. In these embodiments, there is a correlation where the ratio between the central void diameter and the length or width of the interconnections increases as the strength of the structural lattice increases.

It is also believed that a lattice using a repeating radial dodeca-rhombus (hereinafter “RDDR”) unit cell can present a preferable combination of stiffness, strength, fatigue resistance, and conditions for bone ingrowth. In some embodiments, the repeating RDDR lattice is comprised of titanium or a titanium alloy. In FIG. A7 is an isometric view of a single RDDR unit cell B20 containing a full RDDR structure. In FIG. A8 is a side view of a single RDDR unit cell B20 showing the configuration of interconnections when viewed from a lateral direction. A top or bottom view of the RDDR unit cell B20 would be substantially the same as the side view depicted in FIG. A8.

As used herein, an RDDR unit cell B20 is a three-dimensional shape comprised of a central node with radial struts and mirrored struts thereof forming twelve rhombus shaped structures. The node is preferably an octahedron, more specifically a square bipyramid (i.e. a pyramid and inverted pyramid joined on a horizontal plane). Each face of the node is preferably triangular and fixed to each face is a strut comprised of six triangular facets and two end faces. The central axis of each strut can be orthogonal or non-orthogonal relative to the planar surface of each node face. The central axis may follow the centroid of the strut. The RDDR is also characterized by a central node with one strut attached to each face, resulting in a square bipyramid node with eight struts attached.

Examples of node and strut combinations are shown in FIGS. A9-A13. In FIG. A9 is an isometric view of a single node B30 with a single strut B31 attached. The node B30 is a square bipyramid oriented so that two peaks face the top and bottom of a volume B32 defining the bounds of the node B30 and any attached strut(s) B31. The node B30 is oriented so that the horizontal corners are positioned at their closest point to the lateral sides of the volume B32. The strut B31 extends from a node B30 face to the corner of the volume B32 defining the bounds of the node and attached struts. In FIG. A9, the central axis of the strut is 45 degrees above the horizontal plane where the node's planar face is 45 degrees above a horizontal plane.

FIG. A9 also details an octahedron strut B31, where dashed lines show hidden edges of the strut. The strut B31 is an octahedron with an elongate portion of six substantially similar elongate faces and two end faces. The elongate faces B31a, B31b, B31c, B31d, B31e & B31f of the strut B31 define the outer surface of the strut's elongate and somewhat cylindrical surface. Each of the elongate faces B31a, B31b, B31c, B31d, B31e & B31f are isosceles triangles with a first internal angle, angle A, and a second internal angle, angle B, where angle B is greater than angle A. The strut B31 also has two end faces B31f & B31g that isosceles triangles that are substantially similar to one another, having a first internal angle, angle C, and a second internal angle, angle D, and where angle D is greater than angle C. When comparing the internal angles of the elongate faces B31a, B31b, B31c, B31d, B31e & B31f to the end faces B31f & B31g, angle C is greater than angle A.

In FIG. A10 is a side view of the node B30 and strut B31 combination bounded by volume B32. In the side view, the height of the node B30 compared to the height of the cube B32 can be compared easily. In FIGS. A11-A13 are side views of node and strut combinations viewed from a corner of the volume rather than a wall or face, and where the combinations have been modified from FIGS. A9-A10 to change the volumetric density of the resulting unit cell. In FIG. A11, the height of the node B130 has increased relative to the height of the volume B132. Since the distal end of the strut B131 is fixed by the location of a corner of the volume B132, the strut B131 must change its angle relative to its attached node face so that it becomes nonorthogonal. The node B130 and strut B131 combination, where the angle of the strut B131 from a horizontal plane is about 20.6 degrees, would be appropriate for a lattice structure with an elastic modulus of approximately 3 GPa.

In FIG. A12, the height of the node B230 relative to the height of the cube B232 has been increased over the ratio of FIG. A11 to create a node B230 and strut B231 combination that would be appropriate for a lattice structure with an elastic modulus of approximately 4 GPa. As the height of the node B230 increases, the angle between the strut B231 and a horizontal plane decreases to about 18.8 degrees. As the height of the node B230 increases, the size of the node faces also increase so that the size of the strut B231 increases. While the distal end of the strut B231 is fixed to the corner of the volume B232, the size of the distal end increases to match the increased size of the node face to maintain a substantially even strut diameter along its length. As the node and strut increase in size, the volumetric density increases, as does the elastic modulus. In FIG. A13, the height of the node B330 relative to the height of the volume B332 has been increased over the ratio of FIG. A13 to create a node B330 and strut B331 combination that would be appropriate for a lattice structure with an elastic modulus of approximately 10 GPa. In this configuration, the angle B333 between the strut B331 and a horizontal plane decreases to about 12.4 degrees and the volumetric density increases over the previous examples. The single node and strut examples can be copied and/or mirrored to create unit cells of appropriate sizes and characteristics. For instance, the angle between the strut and a horizontal plane could be increased to 25.8 degrees to render a lattice with a 12.3 percent volumetric density and an elastic modulus of about 300 MPa. While a single node and single strut were shown in the examples for clarity, multiple struts may be attached to each node to create an appropriate unit cell.

Adjacent struts extending from adjacent node faces on either the upper half or lower half of the node have an angle from the horizontal plane and a lateral separation angle defined by an angle between the strut directions of adjacent struts. In the MRDD and RDDR structures, adjacent struts have an external edge or face of the elongate portion extending closest to the relevant adjacent strut. The lateral separation angle, as used herein, generally refers to the angle between an external edge or face of the elongate portion of a strut extending closest to the relevant adjacent strut. In some embodiments, a lateral separation angle defined by a line extending between the center of the strut end faces or a line defined by the center of mass of the struts can be used in reference to a similar calculation for an adjacent strut.

The lateral separation angle is the angle between the nearest face or edge of a strut to an adjacent strut. The lateral separation angle can be measured as the smallest angle between the nearest edge of a strut to the nearest edge of an adjacent strut, in a plane containing both strut edges. The lateral separation angle can also be measured as the angle between the nearest face of a strut to the nearest face of an adjacent strut in a plane normal to the two strut faces. In embodiments without defined strut edges or strut faces, the lateral separation angle can be measured as an angle between the nearest portion of one strut to the nearest portion of an adjacent strut. For a unit cell in a cubic volume, as the strut angle from the horizontal plane decreases, the lateral separation angle approaches 90 degrees. For a unit cell in a cubic volume, as the strut angle from the horizontal plane increases, the lateral separation angle approaches 180 degrees. In some embodiments, it is preferable to have a lateral separation angle greater than 109.5 degrees. In some embodiments, it is preferable to have a lateral separation angle of less than 109.5 degrees. In some embodiments, it is preferable to have a lateral separation angle of between and including about 108 degrees to about 156 degrees. In some embodiments, it is more preferable to have a lateral separation angle of between and including 111 degrees to 156 degrees. In some embodiments, it is more preferable to have a lateral separation angle of between and including 108 degrees to 120 degrees. In some embodiments, it is most preferable to have a lateral separation angle of between and including about 111 degrees to 120 degrees. In some embodiments, it is more preferable to have a lateral separation angle of between and including 128 degrees to 156 degrees. In FIG. A14 is a side view, viewed from a corner of the cube B432, of a single node B430 with two adjacent struts B431 & B434 attached and where the lateral separation angle B443 is identified. When measured from the nearest edge of a strut to the nearest edge of an adjacent strut, the lateral separation angle B443 is about 116 degrees.

In some embodiments, a unit cell is built up from multiple sub-unit cells fixed together. In FIG. A15 is an isometric view of an exemplary sub-unit cell comprising a single node and four struts. In FIG. A16 is an isometric view of two sub-unit cells in a stacked formation where the upper sub-unit cell is inverted and fixed to the top of the lower sub-unit cell. In FIG. A17 is an isometric view of eight sub-unit cells stacked together to form a single RDDR unit cell.

In FIG. A15, the node B530 is a square bipyramid, oriented so that the two peaks face the top and bottom of a cubic volume B532. In some embodiments, the volume B532 can be a cuboid volume, a hexahedron volume, an amorphous volume or of a volume with one or more non-orthogonal sides. The peaks refer to the point where four upper faces meet and the point where four lower faces meet. The node B530 is oriented so that the horizontal vertices face the lateral sides of the cubic volume B532. The strut B531 is fixed to a lower face of the node B530 face on its proximate end and extends to the nearest corner of the cubic volume B532 at its distal end. The distal end of the strut B531 can remain fixed to the cubic volume B532 even if the node B530 changes in size to adjust the sub-unit cell properties.

On the lower face of the node B530 opposite the face which strut B531 is fixed, the proximate end of strut B534 is fixed to the node B530. The strut B534 extends to the nearest corner of cubic volume B532 at its distal end. The strut B535 is fixed on its proximate end to an upper node B530 face directed about 90 degrees laterally from the node B530 face fixed to strut B531. The strut B535 extends to the nearest corner of the cubic volume B532 at its distal end. On the upper face of the node B530 opposite the face which strut B535 is fixed, the proximate end of strut B536 is fixed to the node B530. The strut B536 extends to the nearest corner of the cubic volume B532 at its distal end.

In some embodiments, the struts B531 & B534-B536 are octahedrons with triangular faces. The strut face fixed to a node B530 face can be substantially the same size and orientation of the node B530 face. The strut face fixed to the nearest corner of the cube B532 can be substantially the same size as the strut face fixed to the node B530 and oriented on a substantially parallel plane. The remaining six faces can be six substantially similar isosceles triangles with a first internal angle and a second internal angle larger than said first internal angle. The six substantially similar isosceles triangles can be fixed along their long edges to an adjacent and inverted substantially similar isosceles triangle to form a generally cylindrical shape with triangular ends.

When forming a sub-unit cell B540, it can be beneficial to add an eighth node B538 to each corner of the cube B532 fixed to a strut B531 & B534-B536. When replicating the sub-unit cell B540, the eighth node B538 attached to each strut end is combined with eighth nodes from adjacent sub-unit cells to form nodes located between the struts of adjacent sub-unit cells.

In FIG. A16 is a first sub-unit cell B540 fixed to a second sub-unit cell B640 to form a quarter unit cell B560 used in some embodiments. The second sub-unit cell B640 comprises a square bipyramid node B630 is a square bipyramid, oriented so that the two peaks face the top and bottom of a cubic volume. The node B630 is oriented so that the horizontal vertices face the lateral sides of the cubic volume. The strut B635 is fixed to a lower face of the node B630 face on its proximate end and extends to the nearest corner of the cubic volume at its distal end. On the lower face of the node B630 opposite the face which strut B635 is fixed, the proximate end of strut B636 is fixed to the node B630. The strut B636 extends to the nearest corner of cubic volume at its distal end. The strut B634 is fixed on its proximate end to an upper node B630 face directed about 90 degrees laterally from the node B630 face fixed to strut B635. The strut B634 extends to the nearest corner of the cubic volume at its distal end. On the upper face of the node B630 opposite the face which strut B634 is fixed, the proximate end of strut B631 is fixed to the node B630. The strut B631 extends to the nearest corner of the cubic volume at its distal end.

The first sub-unit B540 is used as the datum point in the embodiment of FIG. A16, however, it is appreciated that the second sub-unit cell B640 or another point could also be used as the datum point. Once the first sub-unit cell B540 is fixed in position, it is replicated so that the second sub-unit cell B640 is substantially similar to the first. The second sub-unit cell B640 is rotated about its central axis prior to being fixed on the top of the first unit-cell B540. In FIG. A16, the second sub-unit cell B640 is inverted to achieve the proper rotation, however, other rotations about the central axis can achieve the same result. The first sub-unit cell B540 fixed to the second sub-unit cell B640 forms a quarter unit cell B560 that can be replicated and attached laterally to other quarter unit cells to form a full unit cell.

Alternatively, a full unit cell can be built up by fixing a first group of four substantially similar sub-unit cells together laterally to form a square, rectangle or quadrilateral when viewed from above. A second group of four substantially similar sub-unit cells rotated about their central axis can be fixed together laterally to also form a square, rectangle or quadrilateral when viewed from above. The second group of sub-unit cells can be rotated about their central axis prior to being fixed together laterally or inverted after being fixed together to achieve the same result. The second group is then fixed to the top of the first group to form a full unit cell.

In FIG. A17 is an example of a full unit cell B770 formed by replicating the sub-unit cell B540 of FIG. A15. The cube B532 defining the bounds of the sub-unit cell B540 is identified as well as the node B530 and struts B531 & B534-B536 for clarity. The full unit cell B770 of FIG. A17 can be formed using the methods described above or using variations within the inventive concept.

Each strut extending from the node, for a given unit cell, can be substantially the same length and angle from the horizontal plane, extending radially from the node. At the end of each strut, the strut is mirrored so that struts extending from adjacent node faces form a rhombus shaped opening. Because the struts can be non-orthogonal to the node faces, rhombuses of two shapes emerge. In this configuration, a first group of four rhombuses extend radially from the node oriented in vertical planes. The acute angles of the first group of rhombuses equal twice the strut angle from the horizontal plane and the obtuse angles equal 180 less the acute angles. Also in this configuration is a second group of eight rhombuses extending radially so that a portion of the second group of eight rhombuses fall within the lateral separation angle between adjacent struts defining the first group of four rhombuses. The acute angles of the second group of rhombuses can be about the same as the lateral separation angle between adjacent struts that define the first group of four rhombuses and the obtuse angles equal 180 less the acute angles. The characteristics of a scaffold may also be described by its surface area per volume. For a 1.0 mm×1.0 mm×1.0 mm solid cube, its surface area is 6.0 square mm. When a 1.0 cubic mm structure is comprised of a lattice structure rather than a 100 percent volumetric density material, the surface area per volume can increase significantly. In low volumetric density scaffolds, the surface area per volume increases as the volumetric density increases. In some embodiments, a scaffold with a volumetric density of 30.1 percent would have a surface area of 27.4 square mm per cubic mm. In some embodiments, if the volumetric density was decreased to 27.0 percent, the lattice would have a surface area of 26.0 square mm per cubic mm and if the volumetric density were decreased to 24.0 percent, the lattice would have a surface area of 24.6 square mm per cubic mm.

The MRDD and RDDR structures disclosed herein also have the advantage of an especially high modulus of elasticity for a given volumetric density. When used as a lattice or scaffold, an implant with an adequate modulus of elasticity and a low volumetric density can be achieved. A low volumetric density increases the volume of the implant available for bone ingrowth.

In Table 1, below, are a number of example lattice configurations of various lattice design elastic moduli. An approximate actual elastic modulus was given for each example, representing a calculated elastic modulus for that lattice after going through the manufacturing process. The lattice structures and implants disclosed herein can be designed to a design elastic modulus in some embodiments and to an approximate actual elastic modulus in other embodiments. One advantage of the presently disclosed lattice structures is that the approximate actual elastic modulus is much closer to the design elastic modulus than has been previously achieved. During testing, one embodiment of a lattice was designed for a 4.0 GPa design elastic modulus. Under testing, the lattice had an actual elastic modulus of 3.1 GPa, achieving an actual elastic modulus within 77 percent of the design elastic modulus.

For each lattice design elastic modulus, a volumetric density, ratio of design elastic modulus to volumetric density, surface area in mm², ratio of surface area to volumetric density and ratio of surface area to lattice design elastic modulus is given.

TABLE 1
Table of example lattice structures based on lattice design elastic modulus in GPa

| | | | Ratio of | | | |
|---|---|---|---|---|---|---|
| Lattice | Approx. | | Design | | Ratio of | Ratio of |
| Design | Actual | | Elastic | | Surface | Surface Area |
| Elastic | Elastic | Volumetric | Modulus to | Surface | Area to | to Lattice |
| Modulus | Modulus | Density | Volumetric | Area | Volumetric | Design Elastic |
| (GPa) | (GPa) | (percent) | Density | (mm²) | Density | Modulus |
| 0.3 | 0.233 | 18.5 | 1.6 | 22.5 | 121.5 | 74.9 |
| 3 | 2.33 | 29.9 | 10.0 | 27.5 | 92.2 | 9.2 |
| 4 | 3.10 | 33.4 | 12.0 | 28.8 | 86.4 | 7.2 |
| 5 | 3.88 | 36.4 | 13.8 | 29.9 | 82.2 | 6.0 |
| 6 | 4.65 | 38.8 | 15.5 | 30.7 | 79.1 | 5.1 |
| 7 | 5.43 | 40.8 | 17.2 | 31.3 | 76.9 | 4.5 |
| 8 | 6.20 | 42.1 | 19.0 | 31.8 | 75.4 | 4.0 |
| 9 | 6.98 | 43.2 | 20.8 | 32.1 | 74.3 | 4.0 |

In some of the embodiments disclosed herein, the required strut thickness can be calculated from the desired modulus of elasticity. Using the following equation, the strut thickness required to achieve a particular elastic modulus can be calculated for some MRDD and RDDR structures:

Strut Thickness=(−0.0035*(Ê2))+(0.0696*E)+0.4603

In the above equation, “E” is the modulus of elasticity. The modulus of elasticity can be selected to determine the required strut thickness required to achieve that value or it can be calculated using a preselected strut thickness. The strut thickness is expressed in mm and represents the diameter of the strut. The strut thickness may be calculated using a preselected modulus of elasticity or selected to determine the modulus of elasticity for a preselected strut thickness.

In some embodiments, the unit cell can be elongated in one or more directions to provide a lattice with anisotropic properties. When a unit cell is elongated, it generally reduces the elastic modulus in a direction normal to the direction of the elongation. The elastic modulus in the direction of the elongation is increased. It is desirable to elongate cells in the direction normal to the direction of new bone growth contained within the interconnections, openings and central voids (if any). By elongating the cells in a direction normal to the desired direction of reduced elastic modulus, the shear strength in the direction of the elongation may be increased, providing a desirable set of qualities when designing a structural scaffold. Covarying the overall stiffness of the scaffold may augment or diminish this effect, allowing variation in one or more directions.

In some embodiments, the sub-unit cells may be designing by controlling the height of the node relative to the height of the volume that defines the sub-unit cell. Controlling the height of the node can impact the final characteristics and appearance of the lattice structure. In general, increasing the height of the node increases the strut thickness, increases the volumetric density, increases the strength and increases the elastic modulus of the resulting lattice. When increasing the height of the node, the width of the node can be held constant in some embodiments or varied in other embodiments.

In some embodiments, the sub-unit cells may be designing by controlling the volume of the node relative to the volume that defines the sub-unit cell. Controlling the volume of the node can impact the final characteristics and appearance of the lattice structure. In general, increasing the volume of the node increases the strut thickness, increases the volumetric density, increases the strength and increases the elastic modulus of the resulting lattice. When increasing the volume of the node, the width or height of the node could be held constant in some embodiments.

In Table 2, below, are a number of example lattice configurations of various lattice design elastic moduli. An approximate actual elastic modulus was given for each example, representing a calculated elastic modulus for that lattice after going through the manufacturing process. The lattice structures and implants disclosed herein can be designed to a design elastic modulus in some embodiments and to an approximate actual elastic modulus in some embodiments. For each lattice design elastic modulus, a lattice approximate elastic modulus, a node height, a volumetric density, a node volume, a ratio of node height to volumetric density, a ratio of node height to lattice design elastic modulus and a ratio of volumetric density to node volume is given.

TABLE 2
Table of example lattice structures based on lattice design elastic modulus in GPa

| | | | | | | Ratio of | |
|---|---|---|---|---|---|---|---|
| | Lattice | | | | | Node | |
| Lattice | Approx. | | | | Ratio of | Height to | Ratio of |
| Design | Actual | | | | Node | Lattice | Vol. |
| Elastic | Elastic | Node | Volumetric | Node | Height | Design | Density to |
| Modulus | Modulus | Height | Density | Volume | to Vol. | Elastic | Node |
| (GPa) | (GPa) | (mm) | (percent) | (mm3) | Density | Modulus | Volume |
| 0.30 | 0.23 | 0.481 | 18.5 | 0.0185 | 2.60 | 1.60 | 9.98 |
| 3.00 | 2.33 | 0.638 | 29.9 | 0.0432 | 2.14 | 0.21 | 6.91 |
| 4.00 | 3.10 | 0.683 | 33.4 | 0.0530 | 2.05 | 0.17 | 6.29 |
| 5.00 | 3.88 | 0.721 | 36.4 | 0.0624 | 1.98 | 0.14 | 5.82 |
| 6.00 | 4.65 | 0.752 | 38.8 | 0.0709 | 1.94 | 0.13 | 5.48 |
| 7.00 | 5.43 | 0.776 | 40.8 | 0.0779 | 1.90 | 0.11 | 5.23 |
| 8.00 | 6.20 | 0.793 | 42.1 | 0.0831 | 1.88 | 0.10 | 5.07 |
| 9.00 | 6.98 | 0.807 | 43.2 | 0.0877 | 1.87 | 0.09 | 4.93 |

Some embodiments of the disclosed lattice structures are particularly useful when provided within an elastic modulus range between an including 0.375 GPa to 4 GPa. Some embodiments, more preferably, include a lattice structure with an elastic modulus between and including 2.5 GPa to 4 GPa. Some embodiments include a lattice structure with a volumetric density between and including five percent to 40 percent. Some embodiments, more preferably, include a lattice structure with a volumetric density between and including 30 percent to 38 percent.

The lattice structures disclosed herein have particularly robust loading and fatigue characteristics for low volumetric density ranges and low elastic moduli ranges. Some embodiments of the lattice structures have a shear yield load and a compressive yield load between and including 300 to 15000N in static and dynamic loading up to 5,000,000 cycles at 5 Hz. Some embodiments have a compressive shear strength and an axial load between and including 300 to 15000N in static and dynamic loading up to 5,000,000 cycles at 5 Hz. Some embodiments have a shear strength and an axial load between and including 300 to 15000N in static and dynamic loading up to 5,000,000 cycles at 5 Hz. Some embodiments have a torsional yield load up to 15 Nm.

In one example, the inventive lattice structure has a volumetric density of between and including 32 percent to 38 percent, an elastic modulus between and including 2.5 GPa to 4 GPa and a shear strength and an axial load between and including 300 to 15000N in static and dynamic loading up to 5,000,000 cycles at 5 Hz. Some examples include a first set of substantially homogeneous openings with a width of about 200 μm to 900 μm and a second set of substantially homogenous openings with a width of about 1 to 15 times the width of the first set of openings, where the number of openings in the second set are provided at a ratio of about 1:8 to 1:12 relative to the number of openings in the first set.

The disclosed structures can also have benefits when used in applications where osteointegration is not sought or undesirable. By including a growth inhibiting coating or skin on a structure, the lattice disclosed herein can be used to provide structural support without providing a scaffold for bone growth. This may be desirable when used in temporary implants or medical devices that are intended to be removed after a period of time.

In some embodiments, the present invention includes an implant comprising a body roughness with a leading edge roughness that is comparatively smooth to ease distraction during implantation. The implants of the present invention may also include an optional impact rail feature to accommodate the attachment of a surgical instrument. The body surface can have roughness attributable to the application of a surface treatment or it can be attributable to the properties of the body material or structure. The term “rough” as used herein with regards to a surface characteristic refers to any surface irregularity, however small, that deviates from a perfectly smooth surface. In some embodiments, the roughness can be quantified by Ra, where Ra is the arithmetic average of the absolute profile height deviations from the mean line. In some embodiments, the body Ra is greater than zero. In some embodiments, the body Ra is more than 1 nm. In some embodiments, the body Ra is more than 1 μm. In some embodiments, the body roughness has an Ra value in the nano, micro or macro scale. In some embodiments, the body roughness has multiple Ra values that can fall within the nano, micro and macro scales. In some embodiments, the body roughness has multiple Ra values that fall within each of the nano, micro and macro scales. In some embodiments, the body roughness has multiple Ra values that fall within the micro and macro scales. In some embodiments, the body roughness has multiple Ra values that fall within the nano and macro scales. In some embodiments, the body roughness has multiple Ra values that fall within the nano and micro scales. As used herein, the nano scale tends to refer to a size measurable in nanometers or microns. As used herein, the micro scale tends to refer to a size measurable in microns. As used herein, the macro scale tends to refer to a size measurable in millimeters. In some cases, the surface irregularities can promote bone attachment. Surface irregularities can include projections, lumps and indentations. A rough surface could possess surface irregularities that are visible to the eye or it could possess surface irregularities that are only visible using magnification. Surface irregularities include any deviation from a substantially flat surface and can include irregularities with sharp edges, rounded edges and anything in between. It is understood that various other measures of roughness may be used to achieve the devices and methods disclosed herein.

The leading edge of the present invention can have a roughness level that is described as smooth, however it must only be smooth relative to the body roughness to provide a benefit. Smooth, unless specified otherwise, merely refers to a surface having projections, lumps or indentations of a lower magnitude than that of another surface.

In some aspects, the present invention is directed towards implants that possess body roughness, whether the body roughness is due to a surface treatment applied to the implant or whether the body roughness is due to a material property. Body roughness may be due to the use of a biocompatible lattice structure in an implant, either on the surface or extending below the surface.

Body roughness on an implant is beneficial for providing a surface that promotes bone attachment and/or to provides a scaffold for bone growth. The body roughness that provides these benefits also can cause additional damage during the implantation process because it does not easily distract during insertion. Once the access is made, some bone or soft tissue may remain (deliberately or otherwise) in the space and the leading edge of an implant is used to push the remaining tissue aside as the implant is positioned. When an implant has surface roughness, the remaining tissue tends not to move to the sides of the implant, causing the procedure to take longer and increasing patient risk.

To ease implantation, the present invention can include an implant with body roughness and a comparatively smooth leading edge to distract tissue during implantation and reduce severity in the event of unintentional tissue contact with the leading edge of the device. The exemplary embodiments used in this disclosure are interbody implants, but there are many other implants that would benefit from a smooth leading edge. Leading edge, as used herein, generally refers to the area of an implant that is inserted first into a patient. The leading edge can also refer to another surface of an implant for devices that are designed for rotation during implantation. The leading edge can refer to any surface of a device that distracts tissue during implantation.

In the first exemplary embodiment of an implant 10, the structure of the implant is provided by a body 17. In some embodiments, the body 17 comprises a lattice. The body 17 extends between an upper endplate 18 on the upper surface of the implant and a lower endplate 19 on the lower surface of the implant. The use of a body comprising a lattice and the use of separate endplates is optional. In some embodiments, the body comprises a material with a body roughness Ra of greater than zero. In some embodiments, the body extends to the upper and lower surfaces of the implant. The term endplate, as used herein, refers to an area with a higher volumetric density than the body of an implant and placed on an outer surface of the implant. The specific directional references used to describe the figures are exemplary and are merely used to in reference to the example orientations described herein. Other directional references could be used, such as a directional reference based on an implant's orientation after implantation. For example, the terms upper and lower can refer to the superior and inferior directions when a spinal interbody is implanted in the spine. Front can refer to the end of a spinal interbody implant that is generally inserted first and back can refer to the end of a spinal interbody implant opposite the front. The back end of the exemplary implant 10 is characterized with a threaded opening that can accept the threaded portion of an insertion tool.

In some embodiments, the implant 10 is a posterior lumbar interbody fusion (hereinafter “PLIF”) implant or a transforaminal lumbar interbody fusion (hereinafter “TLIF”) implant. That some exemplary embodiments comprise a PLIF or TLIF implant does not limit the type of devices capable of design or manufacture using the implant features and methods of design and manufacturing disclosed herein. A single implant can be referred to as either a PLIF or TLIF in some embodiments because it is appreciated that PLIF and TLIF implants are often very similar and sometimes indistinguishable. Compared to PLIF implants, TLIF implants may be slightly longer (front to back) and may have a curve in a lateral direction. PLIF implants are generally implanted from a straight posterior approach, where TLIF implants are generally implanted from an angle between the posterior direction and a lateral direction. Both PLIF and TLIF implants may have lordosis.

In the exemplary implant 10, the leading edge comprises an upper nose 11 and lower nose 12 that has a leading edge roughness that is less than the body roughness. While the upper nose 11 and lower nose 12 do not need to be perfectly smooth to provide a benefit during insertion, they should be as smooth as practical and at least less rough than the body roughness. Smoother, when used herein, can refer to a surface that is less rough than another surface or that has a lower roughness, Ra, than another surface.

The leading edge of the implant 10 is further comprised of an optional gap 21 that separates the upper nose 11 from the lower nose 12. The upper and lower surfaces of the gap 21 do not need be horizontal, but merely should provide a separation between the upper nose 11 and lower nose 12. In the exemplary embodiment, the gap 21 is V-shaped when viewed from the side.

The gap 21 provides a level of flexibility between the upper nose 11 and the lower nose 12 during insertion and post-operation. When using a body 17 with an elastic modulus that allows compression when subjected to normal physiological stresses, the gap 21 prevents the leading edge from creating an area of excess rigidity. By allowing the upper nose 11 and the lower nose 12 to move relative to one another, there is a reduced risk of failure when the body 17 is compressed. The body 17 may be compressed during implantation if the space for the implant 10 is less than the height of the implant 10 or post-operation when a patient moves.

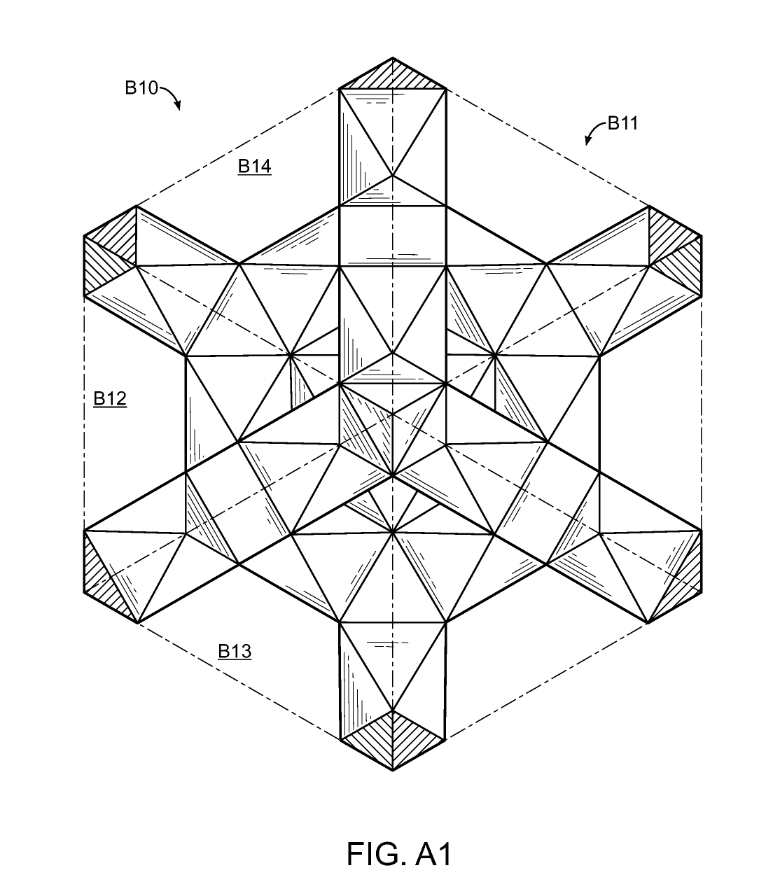

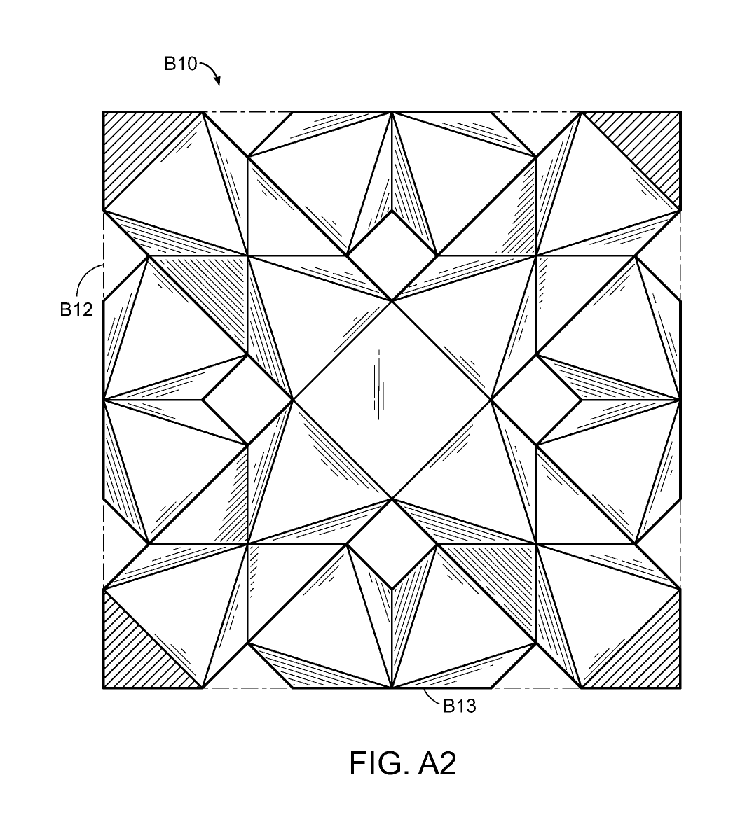

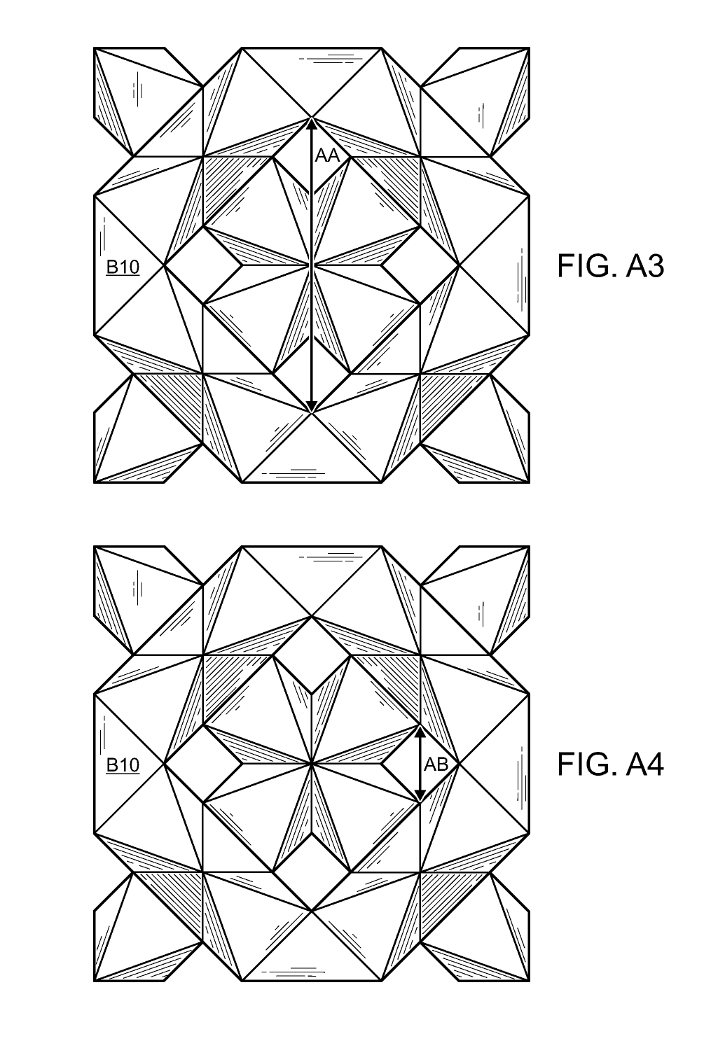

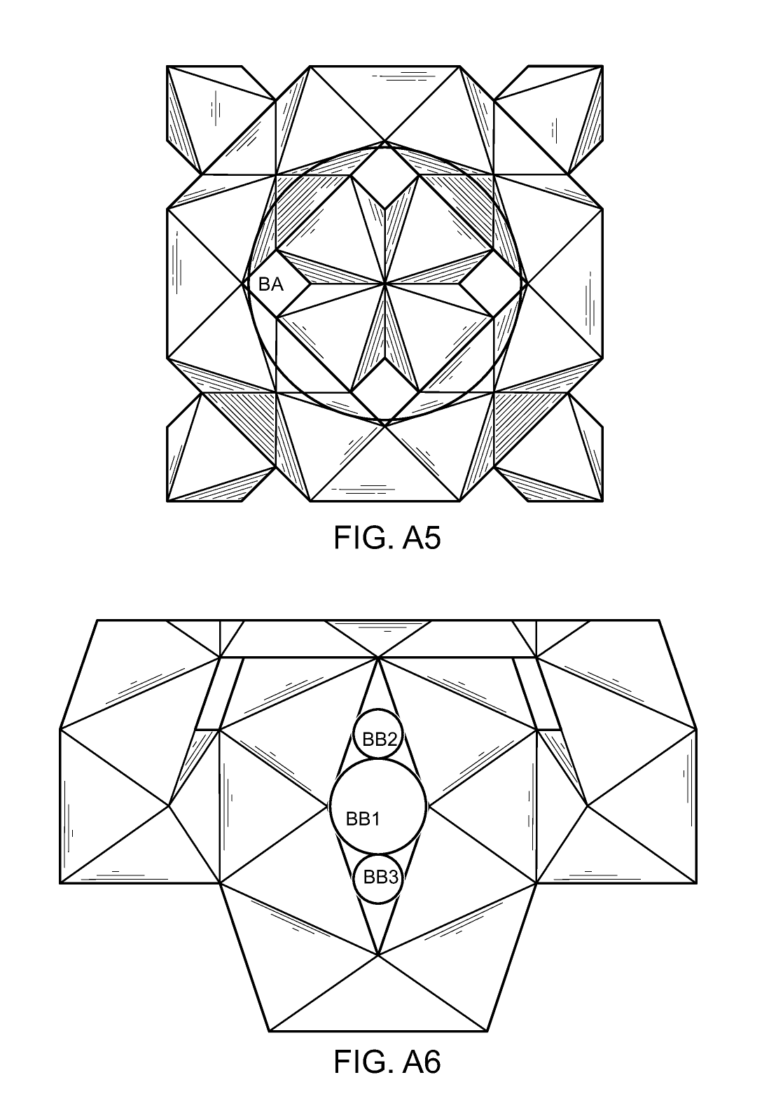

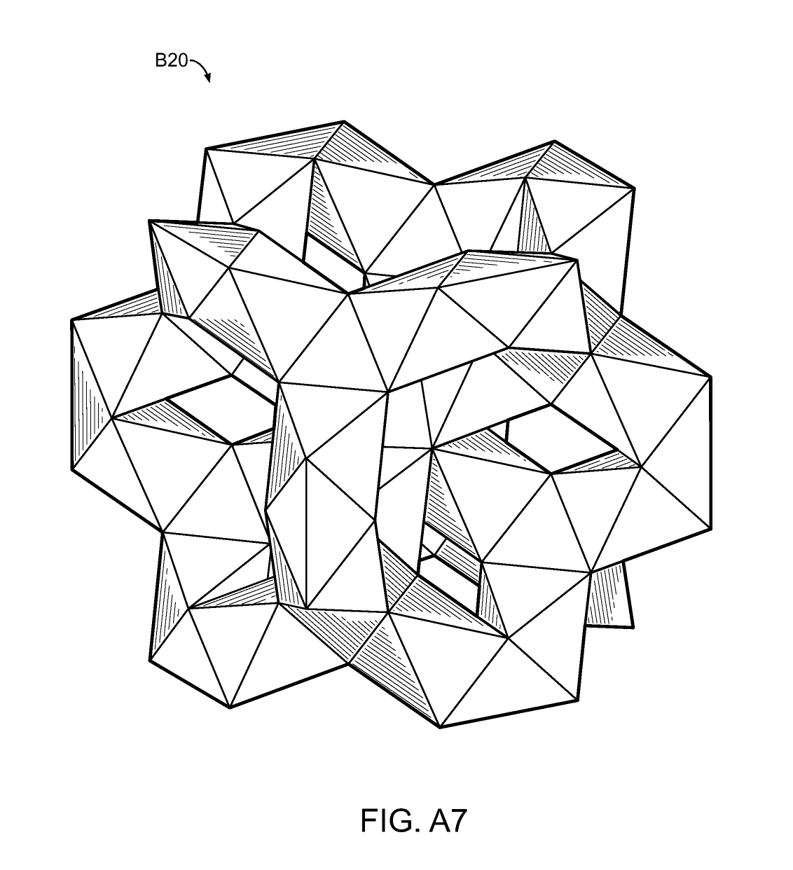

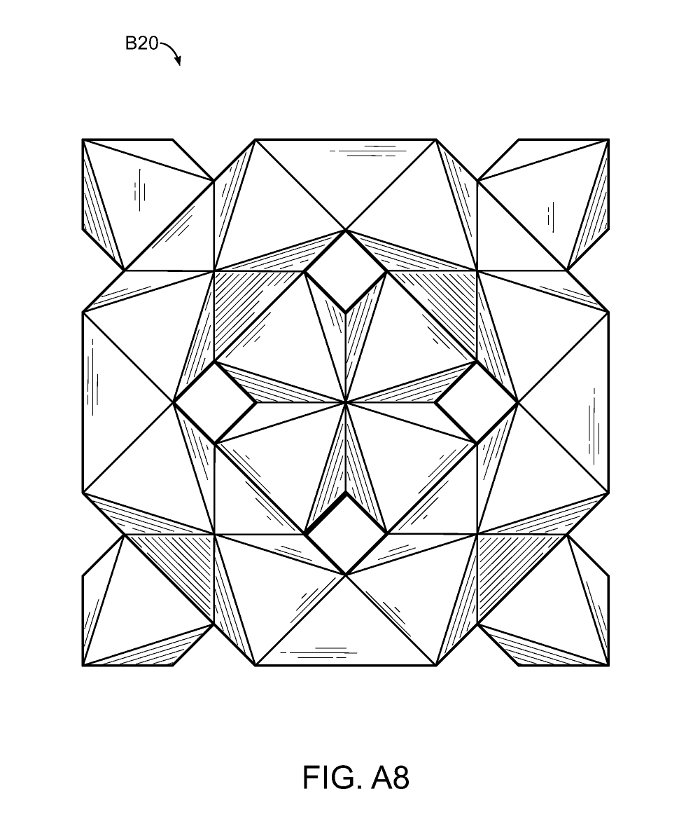

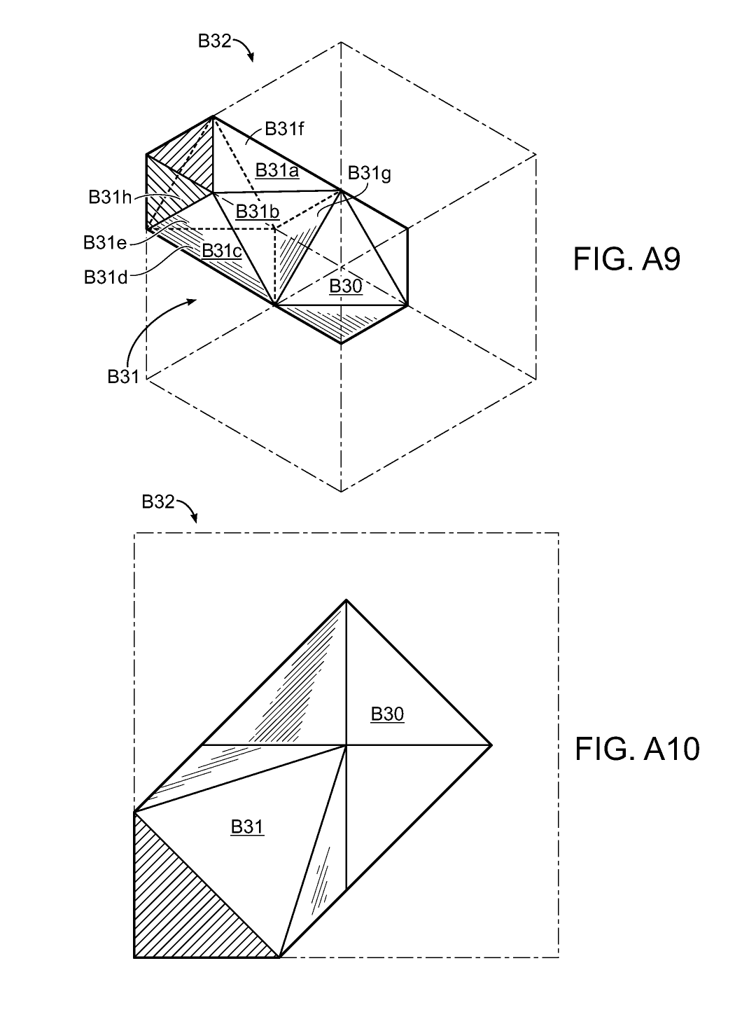

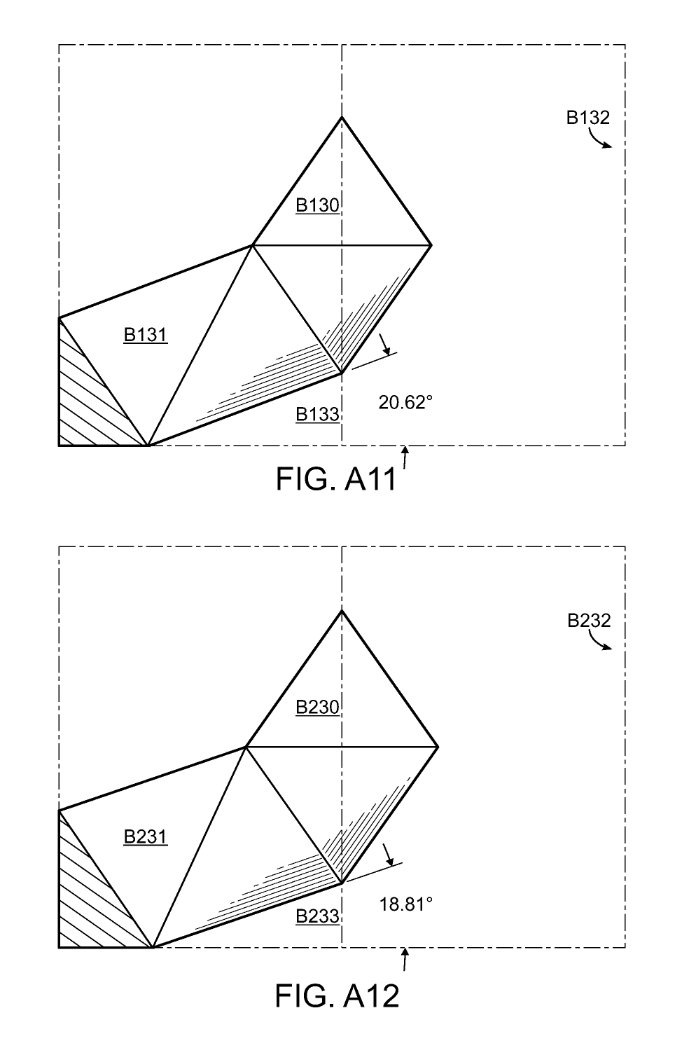

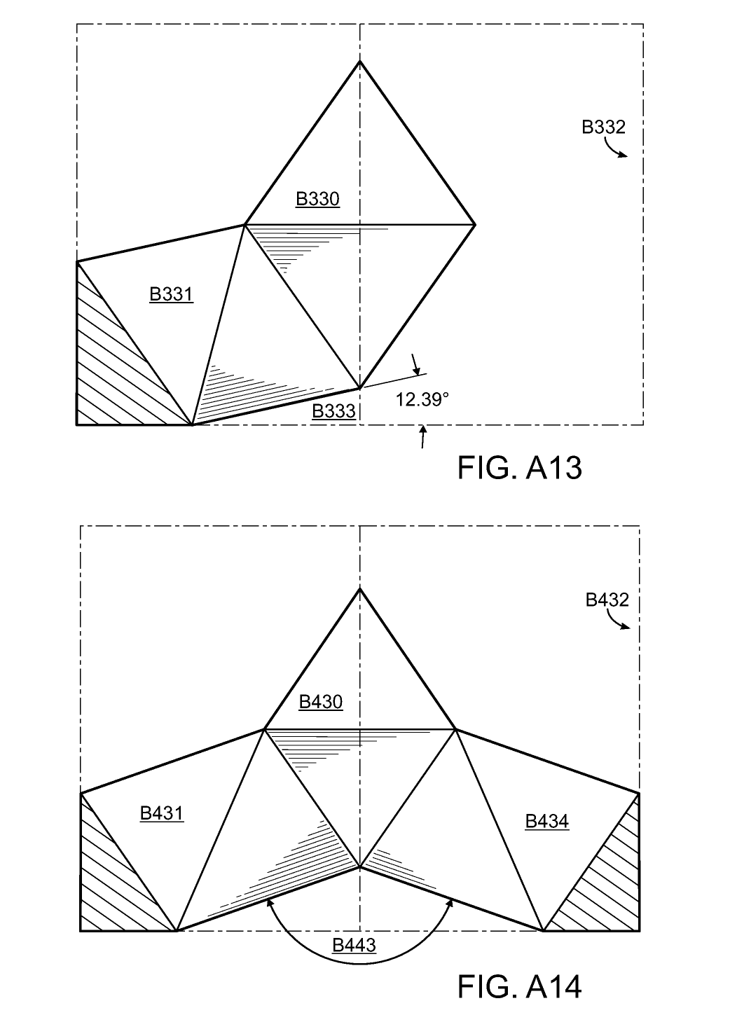

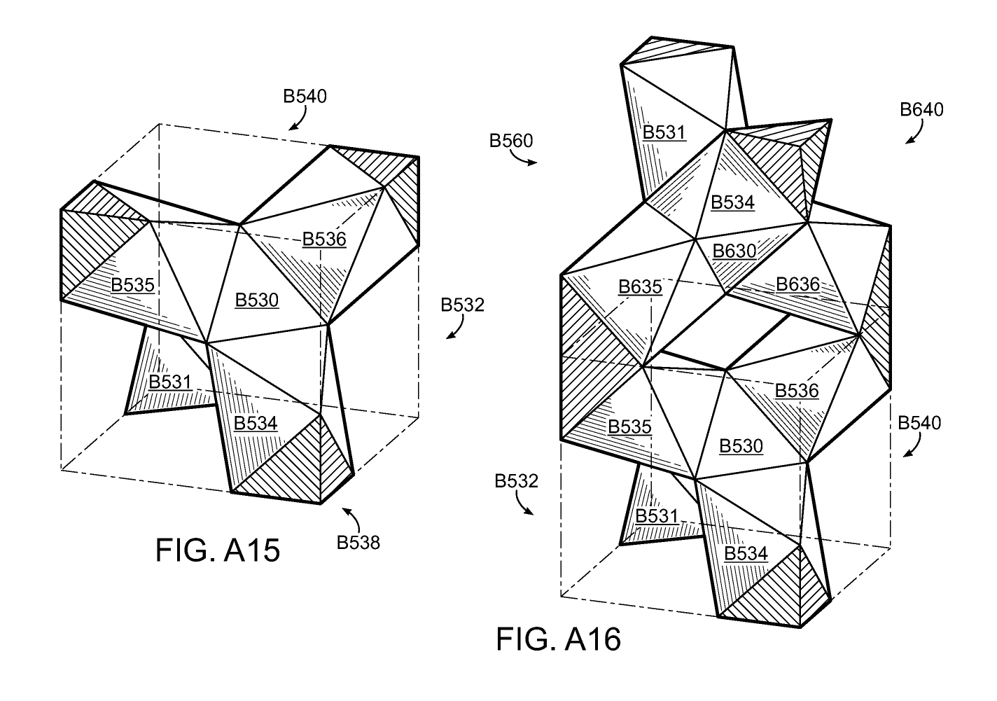

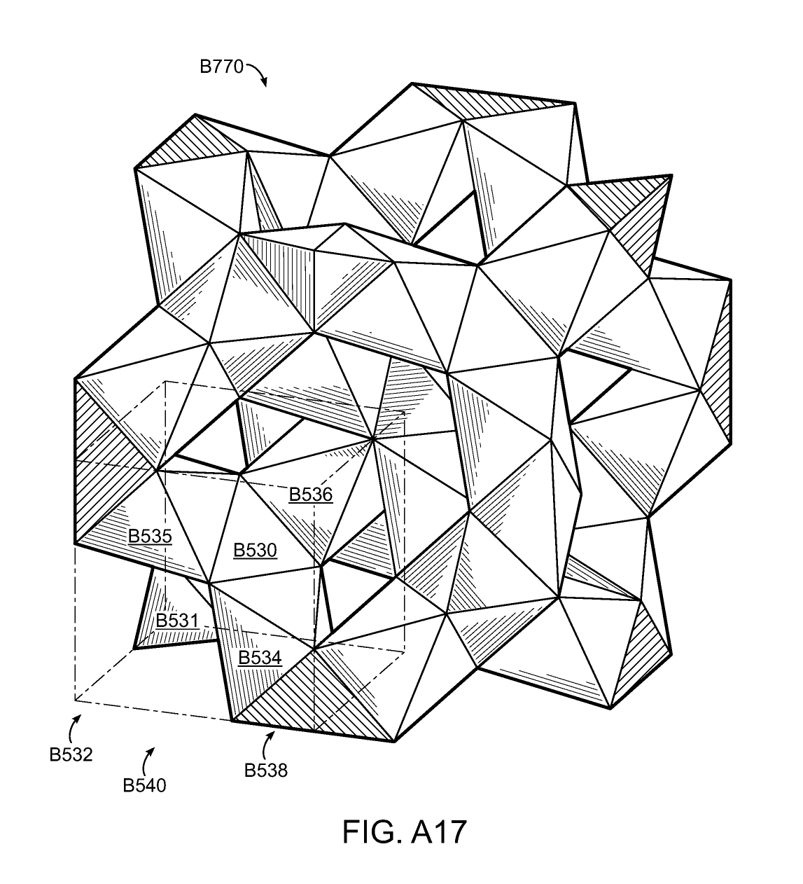

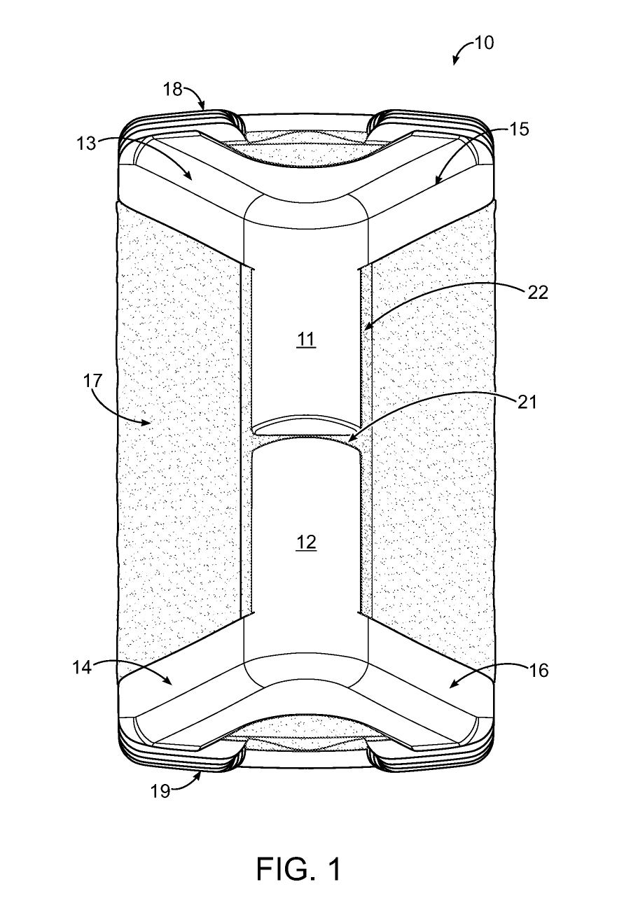

In FIG. 1 is a front view of the implant 10. In the figures disclosed herein, the directions front, side and top are defined based on the orientation of the implant 10 in FIG. 1. The body 17 of the implant 10 is disposed behind the leading edge and can have a volumetric density of less than 100%. The use of a comparatively smooth leading edge is particularly useful in implants with a lattice structure body and a volumetric density of less than 85 percent. In some embodiments, the body 17 comprises a lattice throughout, however, a solid, nonporous body with a body roughness Ra of greater than zero could also be used. Some embodiments have a body with a volumetric density of less than about 50 percent. Some embodiments have a body with a volumetric density of between and including 32 percent to 38 percent. Some embodiments have a body with a porous surface. In some embodiments, the body 17 can have a volumetric density of about 100% and have a body roughness Ra of greater than zero.

The exemplary embodiment of an implant 10 also includes upper nose extensions 13 & 15 and lower nose extensions 14 & 16 that extend to the upper and lower surfaces of the implant 10. In some embodiments, the upper nose extensions 13 & 15 are fixed on one end to the upper endplate 18. In some embodiments, the lower nose extensions 14 & 16 are fixed on one end to the lower endplate 19. When the combined height of the upper nose 11 and lower nose 12 is less than the height of the implant 10, the use of nose extensions 13, 14, 15 & 16 can ease tissue distraction. The outer areas other than the upper nose 11, lower nose 12 and nose extensions 13, 14, 15 & 16 can have a relatively rough surface to promote bone attachment and/or bone ingrowth.

In some embodiments, the nose extensions 13-16 are angled in a taper towards the leading edge of the device. Angling the nose extensions 13-16 in a taper towards the leading edge assists in distracting tissue. The nose extensions 13-16 can be angled in a taper along one or more planes. The nose extensions 13-16 can taper in only one plane, extending from an upper or lower surface of a device down towards a centrally positioned leading edge. The nose extensions 13-16 can also tapper in only one plane by extending from a lateral surface of a device towards a centrally positioned leading edge. The nose extensions 13-16 can taper in more than one plane by extending from an upper or lower and lateral surface of a device towards a centrally positioned leading edge. The nose extensions 13-16 can be substantially straight in some embodiments and curved in some embodiments.

In some embodiments, the angle or curvature of the nose extensions 13-16 can be described relative to a line normal to vertical plane that is normal to the leading edge (hereinafter the “normal line”). In some embodiments, the nose extensions 13-16 are offset by at least 5 degrees from the normal line. In some embodiments, the nose extensions 13-16 are offset by at least 20 degrees from the normal line. In some embodiments, the nose extensions 13-16 are offset laterally and vertically from the normal line. In some embodiments, the nose extensions 13-16 are offset by a greater angle laterally than vertically. In some embodiments, the nose extensions 13-16 are offset by a greater angle vertically than laterally. In some embodiments, the nose extensions 13-16 are offset by about the same angle vertically as laterally.

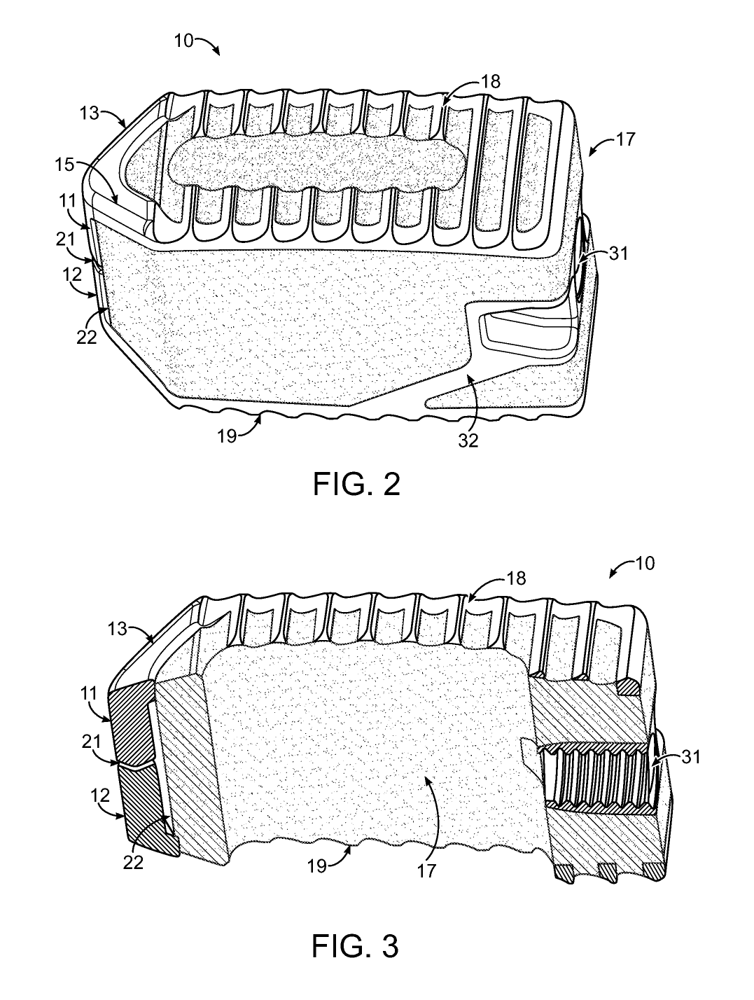

In FIG. 2 is an upper lateral view of the implant 10 and in FIG. 3 is an upper lateral sectioned view of the implant 10. The exemplary implant 10 includes an optional upper endplate 18, an optional lower endplate 19 and optional nose extensions 13, 14, 15 & 16. Omitting the upper and lower endplates 18 & 19 would maximize the ratio of rough surface to comparatively smooth surface. The rear portion of the implant 10 further includes an optional tool engagement area 31 and an optional impact rail feature 32 to distribute force from the tool engagement area 31 to the body 17 or lower endplate 19. The tool engagement area 31 is further comprised of a threaded portion to securely attach a surgical instrument to the implant 10. The tool engagement area 31 and impact rail feature 32 may be omitted if an attachment point for an instrument is not needed.

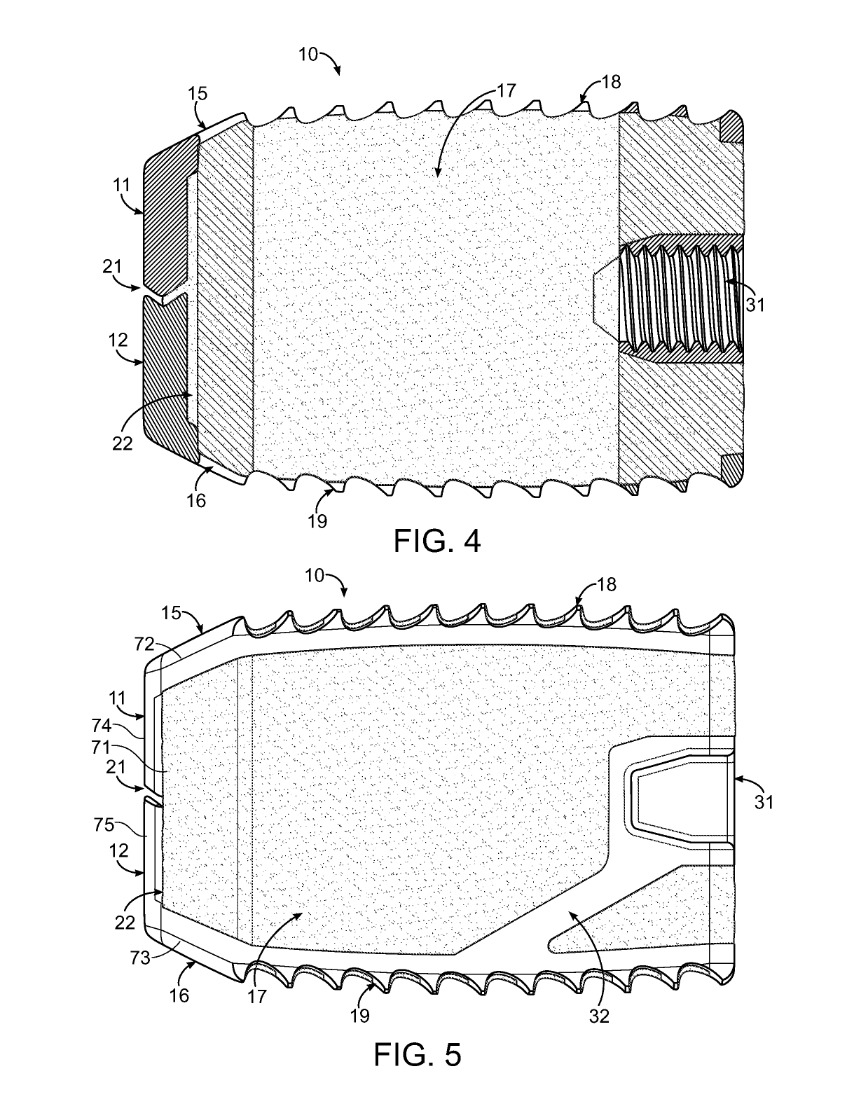

In FIG. 4 is a side sectioned view of the exemplary implant 10. The side sectioned view shows the gap 21 between the upper nose 11 and lower nose 12 as well as the circumferential gap 22 between both upper 11 and lower 12 nose features and the body 17. In FIG. 5 is a side view of the exemplary implant 10, showing an alternate view of the rough surface provided by the body 17. The gap in exemplary implant 10, more clearly visible in FIG. 5, is a v-shaped cut from the lateral direction with comparable surface area on either side of the cut. The gap 21 can be configured in other ways, including but not limited to, two parallel surfaces, a convex conical surface and a corresponding concave conical surface, a v-shaped cut from the front to back direction. In some embodiments, the gap 21 may include more complex cut shapes including curvature(s) in off-plane direction(s), additional linear bend(s) in a perpendicular plane(s), or penetrative feature(s) where one post protrudes into or is enveloped by the other.

The gap 21 can also be characterized by the length of the gap in the axial direction relative to the upper nose feature 11 and lower nose feature 12, which are elongate in about the vertical direction. While the nose features 11 & 12 as about vertical in the implant 10, they may be optionally angled rearward from a vertical plane by zero degrees to 90 degrees. In some embodiments, the gap 21 can be about horizontal. In some embodiments where the nose features 11 & 12 are about vertical, the gap 21 can be at any angle that is not the axial direction of the nose features 11 & 12. The length of the gap can range between zero percent to 85 percent of the implant's overall height. An implant with a low elastic modulus could require a larger gap than an implant with a high elastic modulus to allow the implant to compress without fully compressing the gap. In some embodiments, the length of the gap is between and including 1 percent to 25 percent of the implant's overall height. In some embodiments, the length of the gap is between and including 3 percent to 12 percent of the implant's overall height. In some embodiments, multiple gaps are used with an aggregate total gap distance between zero and 85 percent of the implant's overall height.

The leading edge can comprise multiple shapes and configurations other than the upper and lower nose 11 & 12 in the exemplary implant 10. In some embodiments, the leading edge is elongate in one direction and fixed to the body towards the ends of the elongate ends of the leading edge. In some embodiments, the leading edge is elongate in one direction and fixed to the body towards the middle of the leading edge. In some embodiments, the leading edge comprises multiple segments spaced apart from one another and each fixed to the body, directly or indirectly. In some embodiments, the leading edge comprises multiple segments nested within one another to provide a lockout feature in top to bottom and front to back compression. In some embodiments, the leading edge comprises a single area offset from the normal line on one lateral edge. In some embodiments, the leading edge comprises a single area offset from the normal line on two lateral edges. In some embodiments, the leading edge comprises a single area offset from the normal line on one upper or lower edge. In some embodiments, the leading edge comprises a single area offset from the normal line on one lateral edge and one upper or lower edge. In some embodiments, the leading edge comprises a single area offset from the normal line on two lateral edges and one upper edge and one lower edge. In some embodiments, the leading edge comprises multiple segments offset from the normal line on one or more edge.

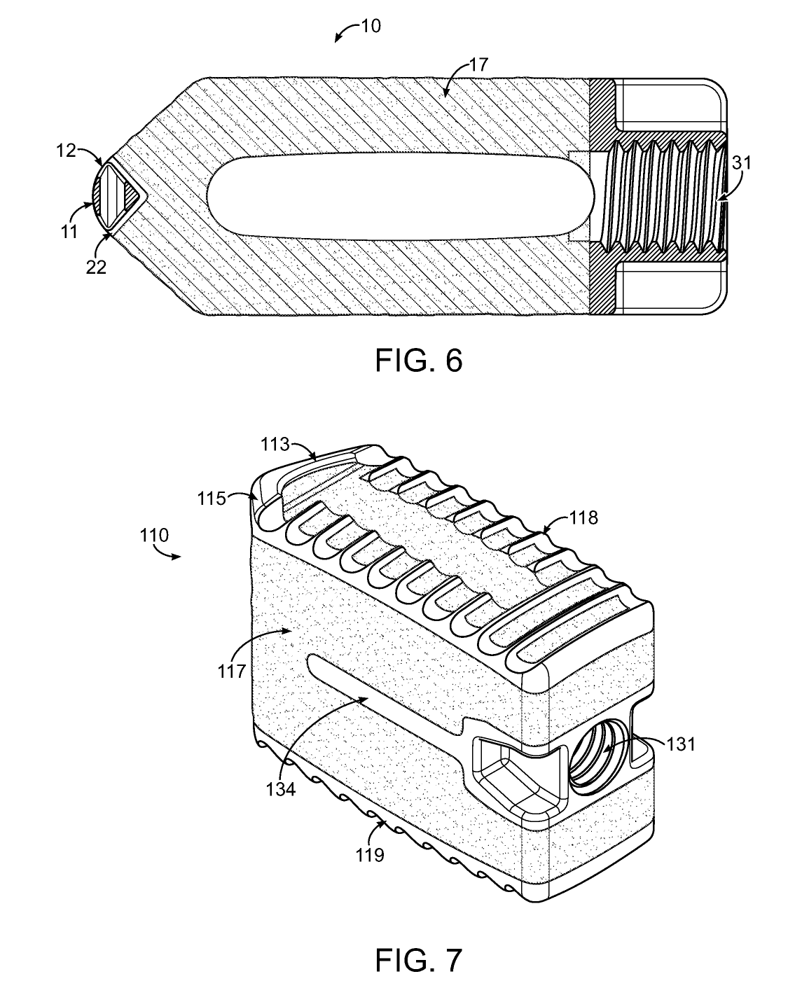

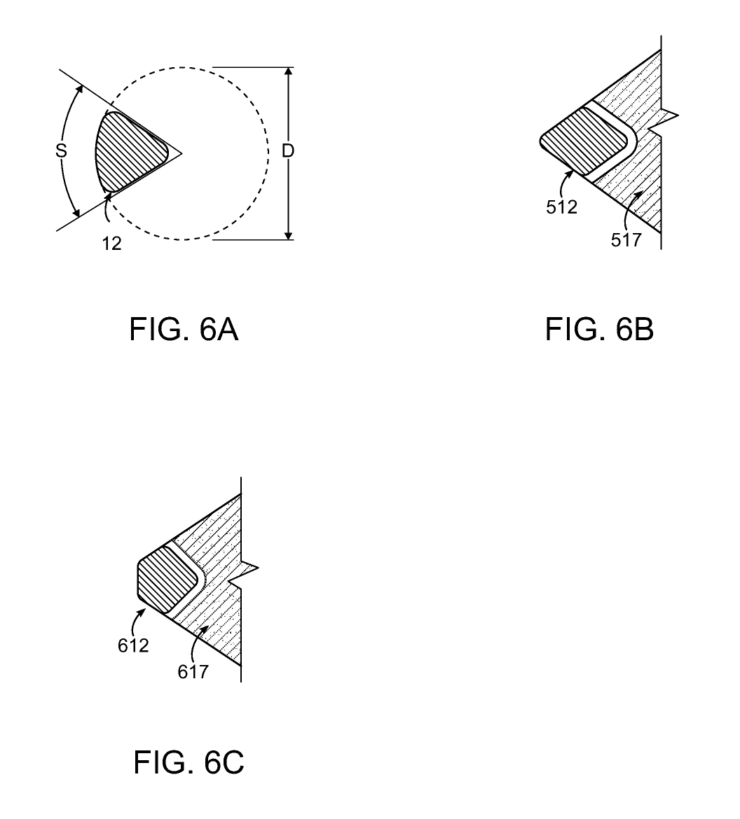

In FIG. 6 is a top sectioned view of the exemplary implant 10 where the implant is sectioned horizontally at its vertical middle. In FIG. 6, the configuration of the nose portion in relationship to the body 17 is visible. The lower nose 12 is visible in this view, including the circumferential gap 22 between the lower nose 12 and the body 17. The use of a circumferential gap 22 between the nose 11 & 12 and the body 17 allows the body 17 to compress independently of the nose 11 & 12. The circumferential gap 22 is particularly useful when the body 17 is comprised of a lattice with a lower elastic modulus than the nose 11 & 12.

In the exemplary implant 10, the implant can be manufactured in a front to back orientation, using external supports. When manufactured in a front to back orientation, external supports can be used in support areas 71, 72, 73, 74 & 75 identified in FIG. 4. Since the opposite side of the implant 10 is substantially the same as the side visible in FIG. 4, there is a support area on the opposite side that mirrors support areas 71, 72 & 73. In some embodiments, the build orientation of the implant 10 can be selected to minimize the need for external supports during the manufacturing process. In some embodiments, the build orientation of the implant 10 can be selected to eliminate the need for external supports during the manufacturing process. In some embodiments, the build orientation of the implant 10 can be selected to minimize the area of the body in contact with external supports during the manufacturing process. In some embodiments, the build surface comprises external supports with a volumetric density less than or equal to the first volumetric density. In some embodiments, the build surface comprises external supports comprising struts with a reduced diameter near their interface with the implant 10 at locations such as support areas 71-75. In some embodiments, where the body comprises a lattice, external supports can connect to the body 17 with struts. In some embodiments using struts to connect external supports to the body 17, the connecting struts can have a smaller diameter than the struts in the body's lattice structure near their interface with the body 17. The leading edge of the upper nose 11 and lower nose 12 of the exemplary implant 10 is rounded and comprises a circular sector centered on the lateral center of the implant 10 and facing forward. A closer view of the lower nose 12 is included in FIG. 6A where the lower nose 12 has been sectioned horizontally at a height of about one third up from the bottom of the implant 10. The circular sector shape of the lower nose 12 can be described based on a sector angle S and a sector diameter D. In some embodiments, the circular sector shape of the lower nose 12 is substantially similar to a circular sector shape of the upper nose 11. In some embodiments, a circular sector defined by the upper nose 11 or lower nose 12 has a sector angle S between and including 1 degree to 225 degrees. In some embodiments, a circular sector defined by the upper nose 11 or lower nose 12 has a sector angle S between and including 25 degrees to 180 degrees. In some embodiments, a circular sector defined by the upper nose 11 or lower nose 12 has a sector angle S between and including 45 degrees to 90 degrees.

The size of a circular sector may be modified based on the dimensions and surface roughness of the remaining portions of the implant. For implants with a smoother outer surface, the circular sector of the nose may be reduced. For implants with a rougher outer surface, the circular sector of the nose may continue in a tangential or other direction from the end of the circular sector. As the length of the implant increases, additional bulleting at the nose can be added while maintaining a sufficient amount of support area. The diameter of the circular sector may also be modified based on the surface roughness of the nose. Implants with a smoother nose surface can use a larger diameter circular sector and implants with a rougher nose surface can use a smaller diameter circular sector. In some embodiments, the circular sector diameter D is about one third the width of the implant. In some embodiments, the circular sector diameter D is between and including 0.15 to 0.9 times the width of the implant. In some embodiments, the circular sector diameter D is between and including 0.2 to 0.5 times the width of the implant. In some embodiments, the circular sector diameter D is between and including 0.2 to 0.35 times the width of the implant. In some embodiments, the circular sector diameter D is between and including 0.36 to 0.44 times the width of the implant.

While the nose portion of the exemplary embodiment follows a circular sector when viewed from above, other configurations may be used for the leading edge. The leading edge, in some examples, has an angled appearance, a flat appearance or a pointed appearance when viewed from above. In FIG. 6B is an example of a lower nose 512 and implant body 517 where the leading edge has a pointed appearance when viewed from above. The view in FIG. 6B is a top sectioned view where the implant has been sectioned horizontally about one third of the way up from the bottom. In FIG. 6C is an example of a lower nose 612 and implant body 617 where the leading edge has a flat appearance when viewed from above. The view in FIG. 6C is a top sectioned view where the implant has been sectioned horizontally about one third of the way up from the bottom.

To ease distraction, the leading edge of the nose 11 & 12 and the nose extensions 13-16 can have a volumetric density that is between that of the body 17 and a value up to and including 100 percent. The leading edge can have the same volumetric density as the nose 11 & 12 and/or the nose extensions 13-16 or a different volumetric density. When the body 17, nose 11 & 12 and nose extensions 13-16 comprise the same material and same lattice structure, the volumetric density of the leading edge of the nose 11 & 12 and nose extensions 13-16 can merely be greater than the volumetric density of the body 17 to provide a benefit. In some examples, the volumetric density of the leading edge of the upper nose 11 and lower nose 12 is between and including 60 percent to 100 percent. The leading edge of the nose extensions 13-16 may have the same volumetric density as the upper nose 11 and lower nose 12, a different volumetric density or a volumetric density that reduces in a gradient in a direction away from the leading edge of the upper nose 11 and lower nose 12. In some embodiments, the volumetric density of the leading edge of the upper nose 11 and the lower nose 12 is between and including 32 percent to 100 percent. In some embodiments, the volumetric density of the leading edge of the upper nose 11 and the lower nose 12 is between and including 10 percent to 80 percent. In some embodiments, where the volumetric density of the upper nose 11 and the lower nose 12 are below 100 percent, a leading edge of a higher volumetric density than the nose 11 & 12 may be fixed to the front of the nose 11 & 12 to provide a smoother leading edge surface.

In some examples, the volumetric density of the upper nose 11 and lower nose 12 is between and including 60 percent to 100 percent. The leading edge of the nose extensions 13-16 may have the same volumetric density as the upper nose 11 and lower nose 12, a different volumetric density or a volumetric density that reduces in a gradient in a direction away from the leading edge. In some embodiments, the volumetric density of the upper nose 11 and the lower nose 12 is between and including 32 percent to 100 percent. In some embodiments, the volumetric density of the upper nose 11 and the lower nose 12 is between and including 10 percent to 80 percent.

Also, to ease distraction, the leading edge of the nose 11 & 12 and the nose extensions 13-16 can have a surface roughness that is less than the body 17 roughness. The surface roughness of the leading edge of the nose 11 & 12 and nose extensions 13-16 can merely be less than the body 17 roughness to provide a benefit. In some embodiments, the leading edge roughness of the upper nose 11 and lower nose 12 is less than 100 percent of the body 17 roughness. In some embodiments, the leading edge roughness of the upper nose 11 and lower nose 12 is between zero percent to 80 percent of the body roughness. In some embodiments, the leading edge roughness of the upper nose 11 and lower nose 12 is between and including 10 percent to 30 percent of the body roughness. In some embodiments, the leading edge of the upper nose 11 and lower nose 12 have a roughness gradient that changes in value across its surface. The nose extensions 13-16 may have the same roughness as the upper nose 11 and lower nose 12, a different roughness or a roughness that changes in a gradient in a direction away from the upper nose 11 and lower nose 12. In some embodiments, the leading edge roughness of the nose extensions 13-16 is less than 100 percent of the body roughness. In some embodiments, the leading edge roughness of the nose extensions 13-16 is between zero percent to 80 percent of the body roughness. In some embodiments, the leading edge roughness of the nose extensions 13-16 is between and including 10 percent to 30 percent of the body roughness.

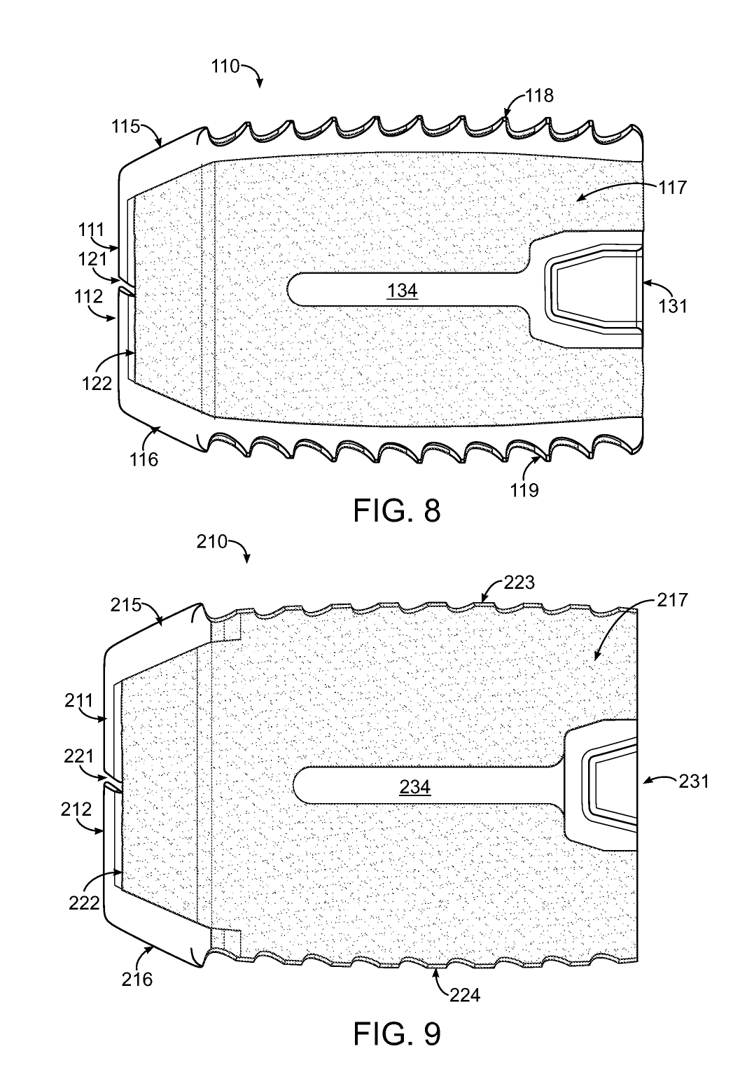

In FIG. 7 is an isometric view of a second exemplary embodiment of the invention as shown on a second implant 110. In FIG. 8 is a side view of the second implant 110. The elements in the alternative embodiments which are substantially the same as the corresponding elements of the first embodiment described are identified with the same numeral. Elements which are similar (but not necessarily identical) in function are denoted by the same numeral plus 100. Directional references used in reference to the second implant 110 are exemplary and used to describe the example orientations disclosed herein.

The second implant 110 is comprises a body 117 with an upper endplate 118 fixed to the top of the body 117 and a lower endplate 119 fixed to the bottom of the body 117. The front of the implant incorporates an upper nose 111 and lower nose 112 that can ease distraction during implantation. Extending between the upper nose 111 and the upper endplate 118 are upper nose extensions 113 & 115. Similar extensions extend from the lower nose 112 to the lower endplate 119, however only lower nose extension 116 is visible in the presented views. Disposed between upper nose 111 and lower nose 112 is a gap 121 that provides a physical gap between the portions of the implant. Directly behind the upper nose 111 and lower nose 112 is a circumferential gap 122 between the aforementioned portions and the body 117. The use of a gap 121 and circumferential gap 122 with the body 117 is an optional feature of the upper and lower nose 111 & 112.

The second implant 110 further comprises an optional tool engagement area 131 and an optional impact rail feature 134. In the second implant 110, the impact rail feature 134 extends largely in the horizontal direction from the back of the implant towards the front, terminating at a location on the side of the body 117. The impact rail feature 134 can distribute impact from the tool engagement area 131 to the body 117 and therefore can be sized or positioned as necessary for the anticipated impact stress. In the second implant 110, the impact rail feature 134 is on the exterior side of the body 117, however, it could be disposed fully within the body 117 or on the edge of the body facing the lumen (i.e. the central vertical opening). The use of an impact rail feature 134 that extends horizontally allows the upper endplate 118 and lower endplate 119 to move independently of one another and prevents the impact rail feature 134 from imparting excess rigidity to the implant.

In FIG. 9 is a third exemplary embodiment of the invention as shown on a third implant 210. The specific directional references used to describe the third implant 210 are exemplary and used to describe the example orientations disclosed herein.

The implant 210 comprises of a body 217 without additional endplates attached to the upper or lower surfaces. The upper 223 and lower surfaces 224 are further characterized by teeth that prevent the expulsion of the implant after insertion. The front of the implant 210 incorporates an upper nose 211 and lower nose 212 to ease distraction during implantation. Extending between the upper nose 211 and upper surface 223 are upper nose extensions 215 disposed on each side of the implant 210. While only upper nose extension 215 is shown in the views, the opposite side can have a substantially similar upper nose extension. Similar extensions extend from the lower nose 212 to the lower surface 224. Only lower nose extension 216 is visible in the presented views, however the opposite side can have a substantially similar lower nose extension. Disposed between upper nose 211 and lower nose 212 is a gap 221 that provides a physical gap between the portions of the implant. Directly behind the upper nose 211 and lower nose 212 is a circumferential gap 222 between the aforementioned portions and the body 217. The use of a gap 221 and circumferential gap 222 with the body 217 is an optional feature of the upper and lower nose 211 & 212.

The third implant 210 is further comprises an optional tool engagement area 231 and an optional impact rail feature 234. In the third implant 210, the impact rail feature 234 extends largely in the horizontal direction from the back of the implant towards the front, terminating at a location on the side of the body 217. The impact rail feature 234 can distribute impact from the tool engagement area 231 to the body 217 and therefore can be sized or positioned as necessary for the anticipated impact stress. In the third implant 210, the impact rail feature 234 is on the exterior side of the body 217, however, it could be disposed fully within the body 217 or on the edge of the body facing the lumen (i.e. the central vertical opening). The use of an impact rail feature 234 that extends horizontally allows the upper surface and lower surface to move independently of one another and prevents the impact rail feature 234 from imparting excess rigidity to the implant.

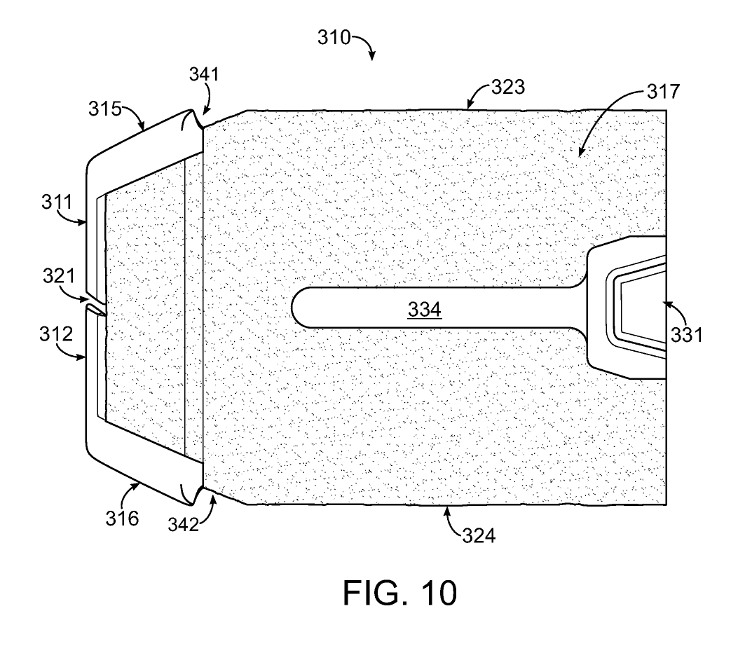

In FIG. 10 is a fourth exemplary embodiment of the invention as shown on a fourth implant 310. The specific directional references are exemplary and used to describe the example orientations disclosed herein.

The fourth implant 310 comprises of a body 317 without additional endplates attached to the upper 323 or lower surfaces 324. The upper 323 and lower surfaces 324 can be substantially flat when viewed from the side, and they can include surface roughness attributed to a surface treatment or due to the material properties of the body 317. The front of the implant 310 incorporates an upper nose 311 and lower nose 312 with a leading edge that can ease distraction during implantation. Extending between the upper nose 311 and upper surface 323 are upper nose extensions 315 disposed on each side of the implant 310. While only upper nose extension 315 is shown in the views, the opposite side can have a substantially similar upper nose extension. Similar extensions extend from the lower nose 312 to the lower surface 324. Only the lower nose extension 316 is visible in the presented views, however the opposite side can have a substantially similar lower nose extension. Disposed between upper nose 311 and lower nose 312 is an optional gap 321 that provides a physical gap between the portions of the implant. Directly behind the upper nose 311 and lower nose 312 is a circumferential gap 322 between the aforementioned portions and the body 317. The use of a gap 321 and circumferential gap 322 with the body 317 is an optional feature of the upper and lower nose 311 & 312.

Between the upper nose extension 315 and the upper surface 323 of the implant 310 is an optional upper anti-expulsion feature 341 that prevents the implant 310 from being displaced once implanted. A similar optional lower anti-expulsion feature 342 is disposed between the lower nose extension 316 and the lower surface 324 of the implant 310. The anti-expulsion features 341 & 342 are shown in FIG. 10 as asymmetrical V-shaped grooves oriented in the lateral direction, however, any type of depression between the nose extensions 315 & 316 and the upper and lower surfaces 323 & 324 of the implant 310 will provide a measure of anti-expulsion benefit. The anti-expulsion features 341 & 342 may also accomplished through the addition of a rise between the nose extensions 315 & 316 and the upper and lower surfaces 323 & 324 of the implant 310. In some embodiments, the anti-expulsion features 341 & 342 have a roughness Ra that is higher than the leading edge roughness or the body roughness.

The fourth implant 310 further comprises an optional tool engagement area 331 and an optional impact rail feature 334. In the fourth implant 310, the impact rail feature 334 extends largely in the horizontal direction from the back of the implant towards the front, terminating at a location on the side of the body 317. The impact rail feature 334 can distribute impact from the tool engagement area 331 to the body 317 and therefore can be sized or positioned as necessary for the anticipated impact stress. In the fourth implant 310, the impact rail feature 334 is on the exterior side of the body 317, however, it could be disposed fully within the body 317 or on the edge of the body facing the lumen (i.e. the central vertical opening). The use of an impact rail feature 334 that extends horizontally allows the upper surface and lower surface to move independently of one another and prevents the impact rail feature 334 from imparting excess rigidity to the implant.

The volumetric density ranges and material properties described for the first exemplary embodiment shown in the first implant 10 can be similarly applied to the alternative embodiments of the implant 110, 210 & 310. Similarly, the sector diameter and sector width ranges stated for the first implant 10 can be applied to the alternative embodiments of the implant 110, 210 & 310.

Another aspect of the present invention is a method of designing and manufacturing implants comprising reduced volumetric density structures. The method of designing and manufacturing implants disclosed herein is particularly useful for implants comprising a lattice, porous or open cell structure, allowing their manufacture without damaging or distorting a portion of the implant that is in contact with the build surface during an additive manufacturing process. Implants comprising lattice structures can be useful to provide a scaffold for bone or tissue growth in the body, but when the first layer of an implant is a lattice structure, damage is highly likely when separating the implant from the build surface after the completion of an additive process. While DMLS and SLS are the focus of the present invention, other appropriate additive production processes include, but are not limited to, selective laser melting, electron beam melting, laminated object manufacturing and directed energy deposition.

While the exemplary embodiments focus on the use of a lattice structure that can provide a scaffold for bone or tissue growth, the methods disclosed herein can be applied to other structures with similar results, including but not limited to, open cell surfaces, closed cell surfaces, closed cell structures, porous surfaces and porous structures. An open cell surface is a layer of open cell material applied or fixed to the surface of an implant. An open cell surface may be one or more cells deep, but does not extend throughout the volume of an implant. A closed cell surface is a layer of closed cell material applied or fixed to the surface of an implant that does not extend throughout the volume of the implant. A closed cell structure is a volume of closed cell material that is similar to an open cell structure or open cell scaffold, but where the cells are not open to one another. A closed cell structure may be accomplished by multiple methods, including the filling of certain cells or through the use of solid walls between the struts of unit cells. A closed cell structure can also be referred to as a cellular structure.

When using an additive process to produce an implant, the use of an open cell structure at or near the surface often results in damage to the surface, especially if the first layer printed is a lattice structure. As used herein, the first layer refers to the layer of material sintered or attached directly to the build surface during the first or first few passes in an additive process. The first layer must conform to the build surface and remain attached during the additive process. If the first layer warps and/or pulls away from the build surface prior to the completion of the additive process, the accuracy of the object would be highly compromised. The first layer also needs to be sufficiently strong to resist damage when the implant is removed from the build surface after the completion of the additive process. If the first layer is overly fragile, it can be damaged when separated. Therefore, it is necessary to have an adequately robust first layer and a secure bond between the first layer and the build surface during the additive process.

This secure bond between the first layer and the build surface becomes problematic as the volumetric density of the first layer decreases and area of the first layer increases. As the volumetric density decreases, at a certain level, rather than breaking away cleanly from the build surface after the additive process, portions of the first layer can stick and deform or cause fractures internal to the implant as the implant is removed. In most circumstances, objects manufactured using an additive process are removed by hand by either pushing the distal end relative to the first layer to generate a torque at the union between the first layer and the build surface or by twisting the object to provide a torsional force at the union. Using either method to remove an implant with a first layer comprising a lattice structure is likely to result in a deformation of that surface. Implants may also be removed from a build surface using a scraping device, but the shear force could potentially damage a first layer comprising a lattice structure. Deformations in the first layer can cause irregularities in the structure near that surface, leaving partial or weakened structures at the surface that could break away in vivo. As the area of the first layer increases, the amount of force necessary to separate the implant from the build surface increases. Therefore, as the area of the first layer increases, the amount of torque necessary to separate the first layer from the build surface increases, thus increasing the possibility of deformation or damage to the implant. While a first layer with a smaller area is desirable to reduce the required removal torque, the first layer must still be adequately sized to bond with the build surface for the duration of the additive process. In an alternate method, implants may be cut from the print bed using electrical discharge machining (EDM) or similar processes, from which the open cell scaffold would also benefit from this invention.

Capable of preventing the deformation of a lattice first layer, the present invention provides a method of design and manufacture that reduces the occurrence of damage when removing lattice structures from the build surface on a machine using an additive process. The present invention can include the step of selecting a build orientation the step of adding features to the first layer to ease removal from the build surface and optimizing the area of the first layer to balance the need for stability during an additive process and the need for ease of separating the device when the additive process is complete.

The build orientation for the implant is selected by taking into account the limitations of the additive process machine, the mechanical loads the implant is expected to experience and optionally by reducing the area of the first layer. Other factors may also be considered in selecting a build orientation, including but not limited to, impact on the isotropy of the construct (prior to heat-treatment for grain unification) for which orientation is typically kept in a direction non-orthogonal to the principle axis of loading; minimize risk of delamination between layers (prior to heat-treatment for grain unification); avoid overhanging features at angles greater than typically 45° (or according to the specific limitations of the additive process machine being used); round and threaded features perform better when built vertically; concentricity is a concern when built vertically; sag will increase further from the test-bed. The build orientation defines the direction in which the implant will be manufactured using the additive process. For instance, if the build orientation of an implant is bottom to top, the bottom surface would be the first layer attached to the build surface and each successive layer would be horizontal to the build platform. The build orientation does not necessarily need to place the implant in an upright position during the additive process, but the build orientation does need to take the orientation of the layers into account. Objects produced using an additive process without supplemental treatment, such as hot isostatic pressing (HIP), are generally weaker in shear in a direction parallel to the build platform than in a direction normal to the build platform. Depending on the final application of the implant, there could be strength considerations that dictate a certain build orientation. Other considerations may also need to be considered when selecting a build orientation, including the limitations of the machine and whether external supports will be used. External supports are required on many DMLS and SLS machines when adding overhanging successive layers with less than a 45-degree angle from the build platform.

Taking the strength requirements for the implant and the potential limitation of the additive process machine into account, the build orientation can be selected. The build orientation selection process may optionally seek to reduce the area of the first layer. Reducing the area of the first layer tends to make it easier to break the bond between the first layer and the build surface after the additive process is complete. With all things being equal, if one build orientation has a first layer with a smaller area, it would be more desirable to use the build orientation with a smaller first layer area than a build orientation with a larger first layer area. While it is desirable to reduce the area of the first layer, the modifications disclosed herein to the first layer would provide a benefit even without limiting the area of the first layer.

With a build orientation selected, the implant is then modified to reduce the occurrence of damage when removed from the build surface. To reduce damage, the first layer, defined as the bottom layer when manufactured in the selected build orientation, is modified by locally increasing its volumetric density, in whole or in part. The modified first layer with an increased volumetric density resists deformation when removed from the build surface and increases stability during the additive process over an equally sized first layer with a lower volumetric density. The first layer also may optionally be modified to reduce its area. Reducing the area of the first layer can further reduce the amount of force needed to remove the implant from the build surface. Depending on the build orientation selected, it may be possible to narrow the implant in the direction of the first layer to reduce the area of the first layer. In some embodiments, the first layer may be reduced by spacing multiple first layer portions apart from one another.

With the build orientation selected and the first layer modified to locally increase volumetric density, the implant can be produced using an additive process. After the implant is manufactured and removed from the build surface, the first layer comprised of material with a higher volumetric density may be left in place or mechanically removed.

The method of designing and manufacturing implants disclosed herein are demonstrated on the exemplary implant 10, however, the method disclosed herein could be applied to other types of implants and implants comprising a different design. The build orientation for the implant 10 was selected as front to back so that the front of 25 the implant 10 faces downward towards the build surface 61, and the back 26 faces upward. The front to back build orientation in the implant 10 example minimizes the area of the first layer, while providing sufficient shear strength and taking the limitations of most additive process machines into account. The first layer of the implant 10 when in the front to back build orientation is the area at the front 25 of the implant 10. In the implant 10 example, the oblique faces on the front 25 of the implant can be angled rearward by at least 45 degrees from the front plane to allow the implant to be produced in this orientation without the use of external supports. The oblique faces on the front 25 of the implant 10 could be angled at less than 45 degrees from the build platform if the additive manufacturing machine allows greater overhand between successive layers or if external supports are used. If a different implant shape is needed, the use of external supports during the additive process could optionally be used.

By selecting a front to back build orientation, the area of the first layer is limited to reduce the chance of deformation when removed from the build surface. The first layer in the implant 10 example still provides adequate stability, in part, because it extends for nearly the full height of the implant. While the implant 10 includes an elongate first layer, many other first layer configurations are possible to provide the benefits described herein. In some embodiments, the first layer is elongate in a direction. In some embodiments, the first layer includes multiple first layer portions spaced apart from one another. In some embodiments, the first layer is a single area that is substantially rectangular, circular or oval.

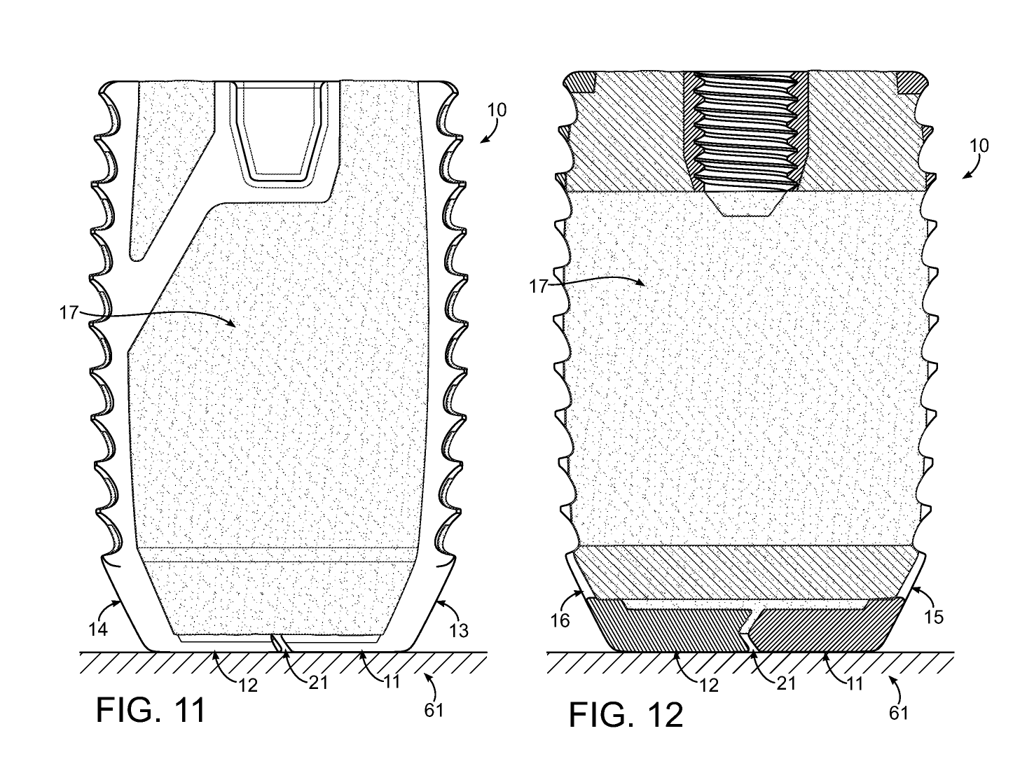

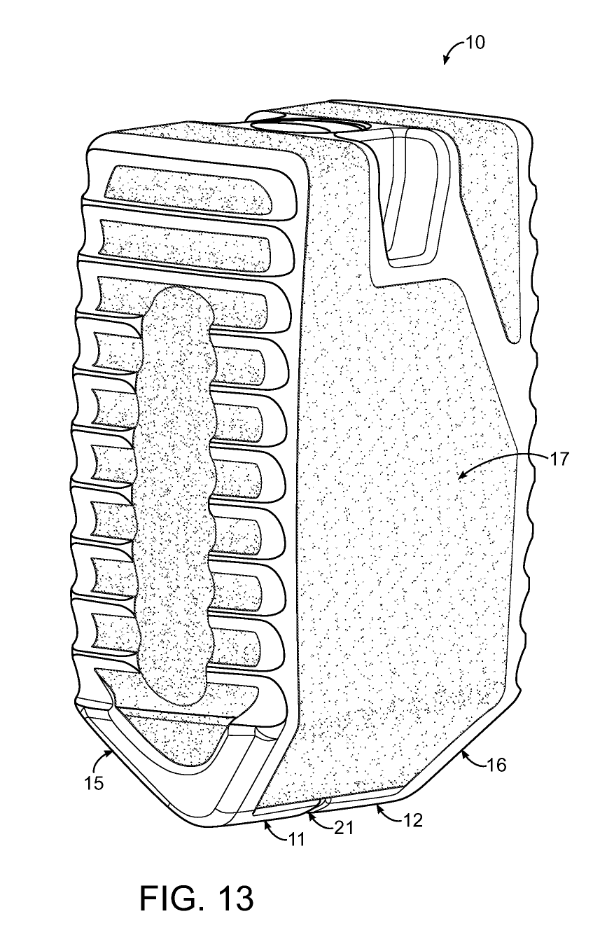

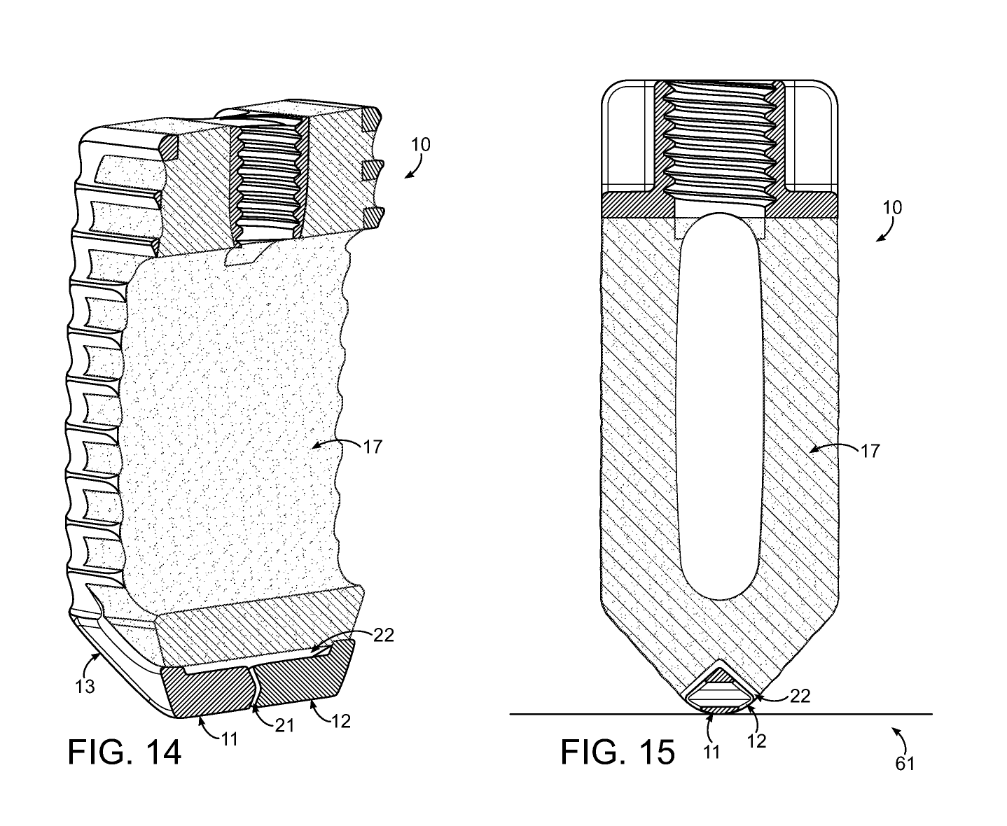

In FIGS. 11 & 12 are views of the implant 10 in a front to back build orientation after manufacturing and prior to being removed from the build surface 61. In the front to back build orientation, the upper nose 11 and lower nose 12 are facing downward prior to being removed from the build surface 61. In FIG. 11, the implant 10 is shown in a side view and attached to the build surface 61. In FIG. 12, the implant 10 is sectioned and shown from the same direction as in FIG. 11. In FIG. 13 is a perspective view of the implant 10 in its front to back build orientation, where the upper nose 11 and lower nose 12 are facing downward. In FIG. 14 is a perspective sectioned view of the implant 10 in its build orientation and in FIG. 15 is a top sectioned view of the implant 10 prior to being removed from the build surface 61.

Because the build orientation of the exemplary implant 10 is a front to back orientation, the first layer comprises the leading edge of the upper nose 11 and lower nose 12. The volumetric density of the leading edge of the upper nose 11 and lower nose 12 were modified in the disclosed method so that they have a volumetric density that is between that of the body 17 and a value up to and including 100%. While the volumetric density of the first layer must only be greater than the volumetric density of the body 17 to provide a benefit, in some embodiments, the volumetric density of the first layer is between and including 60 percent to 100 percent. In some embodiments, the volumetric density of the first layer is between and including 70 percent to 100 percent. In some embodiments, the volumetric density of the first layer is between and including 90 percent to 100 percent. The entire upper nose 11 and lower nose 12 do not need to have the same volumetric density as the first layer to provide a benefit. In some embodiments, the first layer has a different volumetric density than the remainder of the upper nose 11 and lower nose 12. In some embodiments, the volumetric density is reduced in a gradient from the first layer in a rearward direction across the upper nose 11 and lower nose 12.

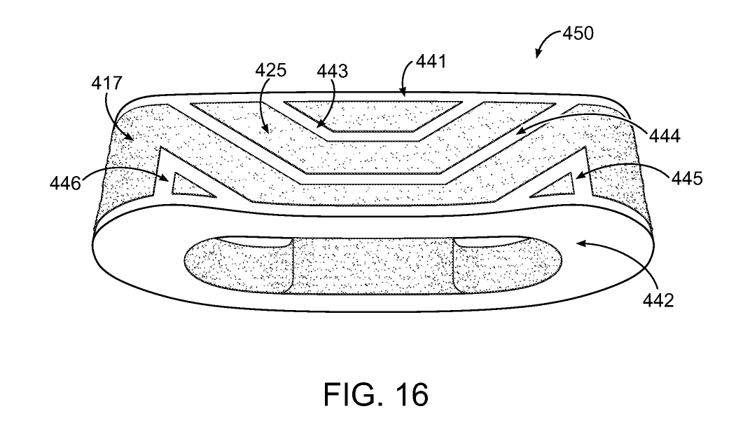

In FIG. 16 is a perspective view of another exemplary implant 450 designed using the method of the present invention after removal from a build surface. The implant 450 is roughly the shape of a rounded rectangle when viewed from above or generally disc shaped, leaving a lateral wall of the implant 450 as the ideal first layer when manufactured using an additive process. The lateral walls of the implant 450 have a significant surface area, making it difficult to manufacture in low volumetric densities because most build orientations have a first layer with a significant area. In some embodiments, the implant 450 is an anterior lumbar interbody fusion (hereinafter “ALIF”) implant so that the front of the implant is the posterior side when implanted in a patient and the back of the implant is the anterior side.

In FIG. 16, the front of the implant 450 is facing upward so that the front side 425 and the bottom end 142 are visible. The implant 450 was manufactured in a front to back build orientation so that the first layer was the front side 425 of the implant. In accordance with the inventive method disclosed herein, the first layer was modified to include localized areas of higher volumetric density to reduce the occurrence of damage to the body 417 upon removal from the build surface.

On the front 425 of the implant 450 are a number of areas of higher volumetric density to aid in the removal of the implant from the build surface after the additive process is complete. The implant 450 comprises an upper first layer support 443, a middle first layer support 444, and two lower first layer supports 445 & 446. The first layer supports 443-446 are configured to reduce the occurrence of damage to the body 417 when removed from a build surface, while minimizing their impact on the elastic modulus of the implant 450 when compressed from the top 441 to the bottom 442. The first layer supports 443-446 are separated across the front 425 of the implant 450 and largely not parallel to the top 441 to bottom 442 direction to prevent their presence from imparting excess rigidity to the body 417 in the top 441 to bottom 442 direction. The first layer supports 443-446 are also thin walled, largely follow the orientation of the cells comprising the body 417 and there are also separations between the first layer supports 443-446 in the top 441 to bottom 442 direction to allow the top 441 and bottom 442 to move independently of one another. The configuration of first layer supports 443-446 is exemplary in nature and can be modified by a person skilled in the art, within the inventive concept disclosed herein.

The volumetric density of the first layer supports 443-446 were modified in the disclosed method so that they have a volumetric density that is between that of the body 417 and a value up to and including 100%. While the volumetric density of the first layer supports 443-446 must only be greater than the volumetric density of the body 417 to provide a benefit, in some embodiments, the volumetric density of the first layer supports is between and including 60 percent to 100 percent. In some embodiments, the volumetric density of the first layer is between and including 70 percent to 100 percent. In some embodiments, the volumetric density of the first layer is between and including 90 percent to 100 percent. The entire upper nose 11 and lower nose 12 do not need to have the same volumetric density as the first layer to provide a benefit. In some embodiments, the first layer has a different volumetric density than the remainder of the upper nose 11 and lower nose 12. In some embodiments, the volumetric density is reduced in a gradient from the first layer in a rearward direction across the upper nose 11 and lower nose 12.

What has been described are implants components for use in implant with surface roughness or a reduced volumetric density and a method of designing and manufacturing implants with a reduced volumetric density. In this disclosure, there are shown and described only exemplary embodiments of the implant components and exemplary embodiments of implants created using the inventive method, but, as aforementioned, it is to be understood that the invention is capable of use in various other combinations and environments and is capable of changes or modifications within the scope of the inventive concept as expressed herein.

1. A medical implant, comprising:
a body with a first volumetric density;
wherein the medical implant has with a front, back, top, bottom, right side and left side;
a first protrusion fixed towards the front of the medical implant;
wherein the first protrusion has a second volumetric density;
wherein the second volumetric density is greater than the first volumetric density; and
wherein the first protrusion has at least one edge with an angle offset from a vertical plane tangent to the first protrusion.

2. The medical implant of claim 1, further comprising a second protrusion with a third volumetric density; wherein the second protrusion has at least one edge with an angle offset from a vertical plane tangent to the second protrusion.

3. The medical implant of claim 2, wherein the first protrusion is fixed to the body at a first anchor point and wherein the second protrusion is fixed to the body at a second anchor point.

4. The medical implant of claim 1, further comprising a first endplate spaced apart from a second endplate, the first end plate being mechanically connected to the second endplate substantially only via the body; wherein the first protrusion is fixed to one of the first endplate or the second endplate.

5. The medical implant of claim 1, further comprising a first endplate spaced apart from a second endplate, the first end plate being mechanically connected to the second endplate substantially only via the body; wherein the first protrusion is fixed to one of the first endplate or the second endplate only via at least one elongate extension.

6. The medical implant of claim 5, further comprising a second protrusion with a third volumetric density; wherein the second protrusion has at least one edge with an angle offset from a vertical plane tangent to the second protrusion; wherein the second protrusion is fixed to one of the first endplate or the second endplate only via at least one elongate extension.

7. The medical implant of claim 6, wherein the first protrusion is elongate in one direction; and wherein the second protrusion is elongate in one direction.

8. The medical implant of claim 7, wherein the first protrusion is spaced apart from the body; and wherein the second protrusion is spaced apart from the body.

9. The medical implant of claim 2, wherein the first protrusion is elongate in one direction; wherein the second protrusion is elongate in one direction; wherein the first protrusion is fixed along an elongate surface to the body; and wherein the second protrusion is fixed along an elongate surface to the body.

10. The medical implant of claim 1, wherein the first protrusion has a leading edge with a fourth volumetric density; and wherein the fourth volumetric density is greater than the second volumetric density.

11. The medical implant of claim 1, wherein the implant is configured to be additively manufactured in a front to back build orientation.

12. The medical implant of claim 1, wherein the first volumetric density is less than 100 percent and wherein the second volumetric density is greater than the first volumetric density.

13. The medical implant of claim 10, wherein the first volumetric density is less than 100 percent and wherein the fourth volumetric density is greater than the first volumetric density.

14. The medical implant of claim 1, wherein the body has a body roughness; wherein the first protrusion has a leading edge roughness; and wherein the body roughness is greater than the leading edge roughness.

15. A medical implant, comprising:
a body with a body roughness;
wherein the medical implant has with a front, back, top, bottom, right side and left side;
a first protrusion, with a first protrusion roughness, fixed towards the front of the medical implant;
wherein the body roughness is greater than the first protrusion roughness; and
wherein the first protrusion has at least one edge with an angle offset from a vertical plane tangent to the first protrusion.

16. The medical implant of claim 15, further comprising a second protrusion with a second protrusion roughness; wherein the second protrusion has at least one edge with an angle offset from a vertical plane tangent to the second protrusion.

17. The medical implant of claim 16, wherein the first protrusion is fixed to the body at a first anchor point and wherein the second protrusion is fixed to the body at a second anchor point.

18. The medical implant of claim 15, further comprising a first endplate spaced apart from a second endplate, the first end plate being mechanically connected to the second endplate substantially only via the body; wherein the first protrusion is fixed to one of the first endplate or the second endplate.

19. The medical implant of claim 15, further comprising a first endplate spaced apart from a second endplate, the first end plate being mechanically connected to the second endplate substantially only via the body; wherein the first protrusion is fixed to one of the first endplate or the second endplate only via at least one elongate extension.

20. The medical implant of claim 19, further comprising a second protrusion with a second protrusion roughness; wherein the second protrusion has at least one edge with an angle offset from a vertical plane tangent to the second protrusion; and wherein the second protrusion is fixed to one of the first endplate or the second endplate only via at least one elongate extension.

21. The medical implant of claim 20, wherein the first protrusion is elongate in one direction; and wherein the second protrusion is elongate in one direction.

22. The medical implant of claim 21, wherein the first protrusion is spaced apart from the body; and wherein the second protrusion is spaced apart from the body.

23. The medical implant of claim 21, wherein the first protrusion is fixed along an elongate surface to the body and wherein the second protrusion is fixed along an elongate surface to the body.

24. The medical implant of claim 15, wherein the implant is configured to be additively manufactured in a front to back build orientation.

25. The medical implant of claim 16, wherein the body roughness is greater than the second protrusion roughness.

26. The medical implant of claim 16 wherein the first protrusion roughness is about the same as the second protrusion roughness.

27. The medical implant of claim 15, wherein the body has a first volumetric density; wherein the first protrusion has a second volumetric density; and wherein the second volumetric density is greater than the first volumetric density.

28. The medical implant of claim 21, wherein the first protrusion has a front and a back; wherein the back of the first protrusion is oriented towards the front of the body; and wherein the front of the first protrusion comprises a circular sector when viewed from above.

29. The medical implant of claim 28, wherein the circular sector has a sector angle, S, of between and including 1 degree to 225 degrees.

30. The medical implant of claim 28, wherein the circular sector has a sector angle, S, of between and including 25 degrees and 180 degrees.

31. The medical implant of claim 28, wherein the circular sector has a circular sector diameter, D, of between and including 0.15 times to 0.9 times a width of the medical implant.

32. The medical implant of claim 28, wherein a surface of the circular sector has a volumetric density that is greater than the remainder of the first protrusion.

33. The medical implant of claim 15, wherein the first protrusion roughness is between 0 percent and 80 percent of the body roughness.

34. The medical implant of claim 17, wherein the first anchor point is spaced apart from the second anchor point.

35. The medical implant of claim 17, wherein the first protrusion is spaced apart from the second protrusion.
